(19) United States
(12) Patent Application Publication
(10) Pub. No.: US 2023/0024972 A1
(43) Pub. Date: Jan. 26, 2023

(54) BAKUCHINOYL RETINOATE AND METHODS OF USE THEREOF

(71) Applicant: Phyto Tech Corp d/b/a/ Blue California Co, Rancho Santa Margarita, CA (US)

(72) Inventors: KATHERINE Joyce OGLESBY, Rancho Santa Margarita, CA (US); Manuel G. VENEGAS, West Chester, OH (US); Suizhou YANG, North Chelmsford, MA (US); Oliver YU, Lexington, MA (US)

(73) Assignee: Phyto Tech Corp d/b/a/ Blue California Co, Rancho Santa Margarita, CA (US)

(21) Appl. No.: 17/843,617

(22) Filed: Jun. 17, 2022

Related U.S. Application Data
(60) Provisional application No. 63/216,299, filed on Jun. 29, 2021.
(60) Provisional application No. 63/212,363, filed on Jun. 18, 2021.

(51) Int. Cl. C07C 69/76; A61K 8/37; A61Q 19/08

(52) U.S. Cl. CPC C07C 69/76; A61K 8/37; A61Q 19/08

(57) ABSTRACT

The present disclosure provides the compound:

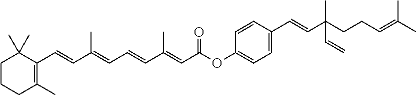 [SMILES: C=CC(C)(/C=C/c1ccc(OC(=O)/C=C(C)/C=C/C=C(C)/C=C/C2=C(C)CCCC2(C)C)cc1)CCC=C(C)C]

and geometric isomers and/or stereoisomers thereof, or a mixture of isomers thereof. The present disclosure also provides compounds having said structure for use in treating symptoms associated with skin ageing and promoting skin health and appearance.

### BACKGROUND OF THE INVENTION

### Field of Invention

The present disclosure provides bakuchinoyl retinoate and geometric isomers and/or stereoisomers thereof. The present disclosure also provides methods of treating symptoms associated with skin ageing and methods of promoting skin health and appearance, comprising administering topical compositions comprising bakuchinoyl retinoate.

### Background

Human skin naturally changes as a result of ageing and exposure to various external elements such as sunlight, abrasives, and chemicals. For example, loss of elasticity, development of wrinkles and/or lines, increased pore size, cracking, flaking, uneven pigmentation, textural irregularities, and dyspigmentation are changes to the skin that can result from the ageing process.

Currently, many topical treatments designed to reduce or ameliorate these changes to the skin utilize retinoid compounds. However, administration of retinoids can result in significant side effects, such as cutaneous erythema, pruritus, peeling, stinging or burning, and sensitivity. Accordingly, there is a need to develop skin therapies comprising alternative active agents that cause fewer or no side effects.

### BRIEF SUMMARY OF THE INVENTION

In one aspect, the disclosure provides bakuchinoyl retinoate, and geometric isomers and/or stereoisomers thereof, and mixtures of geometric isomers and/or stereoisomers, collectively referred to herein as a “Compound of the Disclosure”.

In another aspect, the disclosure provides a composition comprising a Compound of the Disclosure and one or more pharmaceutically, dermatologically, or cosmetically acceptable carriers and/or excipients.

In another aspect, the composition further comprises an effective amount of one or more skin-protective or treatment ingredients, e.g. antioxidants, sunscreen actives, skin lightening actives, exfoliants, anti-acne actives, vitamins, anti-inflammatory agents, self-tanning agents, moisturizers, emollients, humectants, or compatible solutes, or a combination thereof.

In another aspect, the composition is formulated for topical administration.

In another aspect, the composition is formulated as an eye cream or serum, an anti-wrinkle cream or serum, a moisturizing cream, a face wash, a face mask, or a cosmetic.

In another aspect, the disclosure provides a method of improving the common signs of cutaneous facial ageing, e.g., loss of elasticity, development of wrinkles and/or lines, increased pore size, cracking, flaking, uneven pigmentation, textural irregularities, or dyspigmentation, or a combination thereof, in a subject, the method comprising administering to the subject an effective amount of a Compound of the Disclosure, or a composition thereof.

In another aspect, the disclosure provides a method of promoting or maintaining skin health and appearance in a subject, the method comprising administering to the subject an effective amount of a Compound of the Disclosure, or a composition thereof.

In another aspect, the disclosure provides a method of preventing, ameliorating, or reducing the loss of softness and elasticity in the skin of a subject, the method comprising administering to the subject an effective amount of a Compound of the Disclosure, or a composition thereof.

In another aspect, the disclosure provides a method of preventing, ameliorating, or reducing the presence of fine lines and/or wrinkles in the skin of a subject, the method comprising administering to the subject an effective amount of a Compound of the Disclosure, or a composition thereof.

In another aspect, the disclosure provides a method of preventing, ameliorating, or reducing the presence of dark spots in the skin of a subject, the method comprising administering to the subject an effective amount of a Compound of the Disclosure, or a composition thereof.

In another aspect, the disclosure provides a method of promoting or maintaining collagen production in the skin of a subject, the method comprising administering to the subject an effective amount of a Compound of the Disclosure, or a composition thereof.

In another aspect, the disclosure provides a composition comprising a Compound of the Disclosure and a pharmaceutically, dermatologically, or cosmetically acceptable carrier and/or excipient for use in improving the common signs of cutaneous facial ageing in a subject.

In another aspect, the disclosure provides a composition comprising a Compound of the Disclosure and a pharmaceutically, dermatologically, or cosmetically acceptable carrier and/or excipient for use in promoting or maintaining skin health and appearance in a subject.

In another aspect, the disclosure provides a composition comprising a Compound of the Disclosure and a pharmaceutically, dermatologically, or cosmetically acceptable carrier and/or excipient for use in preventing, ameliorating, or reducing the loss of softness and elasticity in the skin of a subject.

In another aspect, the disclosure provides a composition comprising a Compound of the Disclosure and a pharmaceutically, dermatologically, or cosmetically acceptable carrier and/or excipient for use in preventing, ameliorating, or reducing the presence of fine lines and/or wrinkles in the skin of a subject.

In another aspect, the disclosure provides a composition comprising a Compound of the Disclosure and a pharmaceutically, dermatologically, or cosmetically acceptable carrier and/or excipient for use in preventing, ameliorating, or reducing the presence of dark spots in the skin of a subject.

In another aspect, the disclosure provides a composition comprising a Compound of the Disclosure and a pharmaceutically, dermatologically, or cosmetically acceptable carrier and/or excipient for use in promoting or maintaining collagen production in the skin of a subject.

In another aspect, the disclosure provide a Compound of the Disclosure for use in improving the common signs of cutaneous facial ageing in a subject.

In another aspect, the disclosure provide a Compound of the Disclosure for use in promoting or maintaining skin health and appearance in a subject.

In another aspect, the disclosure provide a Compound of the Disclosure for use in preventing, ameliorating, or reducing the loss of softness and elasticity in the skin of a subject.

In another aspect, the disclosure provide a Compound of the Disclosure for use in preventing, ameliorating, or reducing the presence of fine lines and/or wrinkles in the skin of a subject.

In another aspect, the disclosure provide a Compound of the Disclosure for use in preventing, ameliorating, or reducing the presence of dark spots in the skin of a subject.

In another aspect, the disclosure provide a Compound of the Disclosure for use in promoting or maintaining collagen production in the skin of a subject.

In another aspect, the disclosure provides a use of a Compound of the Disclosure for the manufacture of a medicament for use in improving the common signs of cutaneous facial ageing in a subject.

In another aspect, the disclosure provides a use of a Compound of the Disclosure for the manufacture of a medicament for use in promoting or maintaining skin health and appearance in a subject.

In another aspect, the disclosure provides a use of a Compound of the Disclosure for the manufacture of a medicament for use in preventing, ameliorating, or reducing the loss of softness and elasticity in the skin of a subject.

In another aspect, the disclosure provides a use of a Compound of the Disclosure for the manufacture of a medicament for use in preventing, ameliorating, or reducing the presence of fine lines and/or wrinkles in the skin of a subject.

In another aspect, the disclosure provides a use of a Compound of the Disclosure for the manufacture of a medicament for use in preventing, ameliorating, or reducing the presence of dark spots in the skin of a subject.

In another aspect, the disclosure provides a use of a Compound of the Disclosure for the manufacture of a medicament for use in promoting or maintaining collagen production in the skin of a subject.

In another aspect, the disclosure provides methods of making a Compound of the Disclosure.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

A Compound of the Disclosure is a dual-function skin care product comprising bakuchiol, or an isomer thereof, and retinoic acid, or an isomer thereof, coupled together to give the corresponding ester as illustrated in Scheme 1.

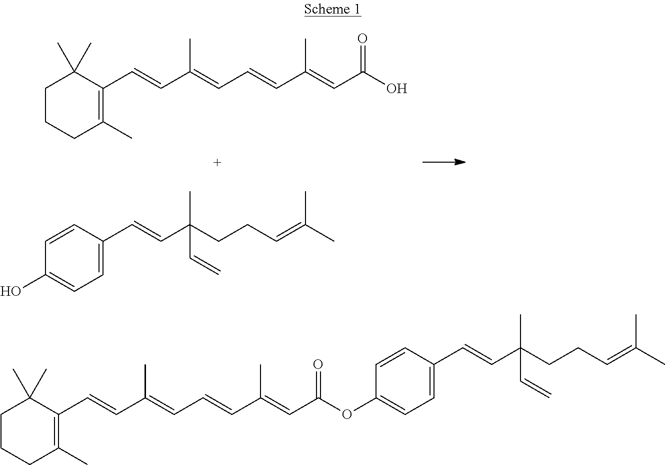 [SMILES: CC1=C(/C=C/C(C)=C/C=C/C(C)=C/C(=O)O)C(C)(C)CCC1 ; C=CC(C)(/C=C/c1ccc(O)cc1)CCC=C(C)C ; C=CC(C)(/C=C/c1ccc(OC(=O)/C=C(C)/C=C/C=C(C)/C=C/C2=C(C)CCCC2(C)C)cc1)CCC=C(C)C]

Without wishing to be bound by any particular theory, bakuchinoyl retinoate acts as a prodrug, e.g., it is metabolized following administration to a subject, to deliver an effective amount of bakuchiol and/or an effective amount of retinoic acid to a subject without the side-effects associated with administering each agent to the subject as separate agents.

A Compound of the Disclosure can be used, for example, to improve the common signs of cutaneous facial ageing in a subject, to promote or maintain skin health and appearance in a subject, to prevent, ameliorate, or reduce the loss of softness and elasticity, presence of fine lines and/or wrinkles, or presence of dark spots in the skin of a subject, or to promote or maintain collagen production in the skin of a subject.

In one embodiment, a Compound of the Disclosure is a compound having the structure:

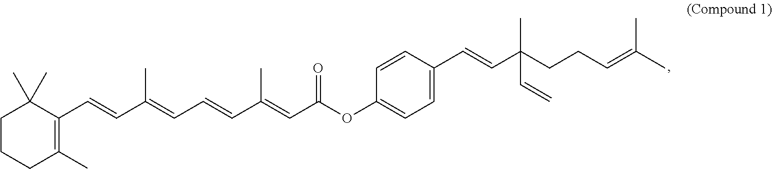 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of geometric isomers and/or stereoisomers thereof.

In another embodiment, a Compound of the Disclosure is a compound of the structure:

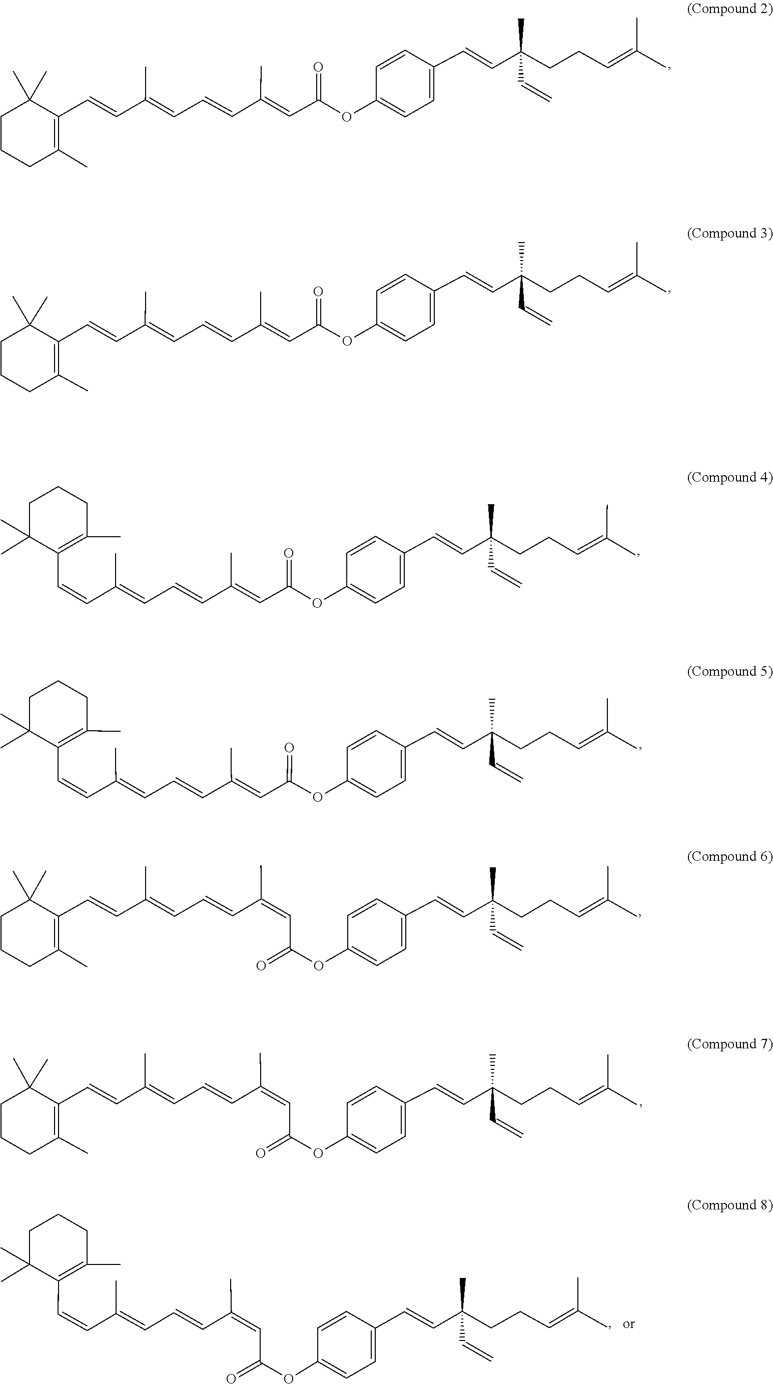

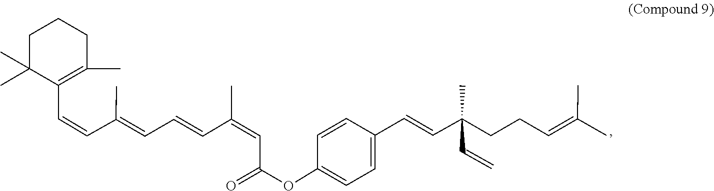

or a mixture thereof.

In another embodiment, a Compound of the Disclosure is the compound with the structure:

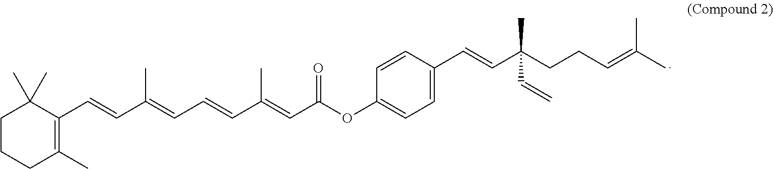

A Compound of the Disclosure contains an asymmetric carbon atom and may thus give rise to enantiomers, diastereomers, and other stereoisomeric forms. Unless specified otherwise, the present disclosure encompasses the use of all such possible forms, as well as their racemic and resolved forms and mixtures thereof. The individual enantiomers can be separated according to methods known in the art in view of the present disclosure. A Compound of the Disclosure also contains multiple olefinic double bonds or other centers of geometric asymmetry and, unless specified otherwise, the present disclosure encompasses the use of all such possible geometric forms and mixtures thereof. For example, it is intended that a Compound of the Disclosure includes both E and Z geometric isomers. All tautomers are also encompassed by the present disclosure.

As used herein, the term “stereoisomers” is a general term for all isomers of an individual molecule that differ only in the orientation of their atoms in space. It includes enantiomers and isomers of compounds with more than one chiral center that are not mirror images of one another (diastereomers).

The term “chiral center” or “asymmetric carbon atom” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image compound rotates the plane of polarized light in the opposite direction.

The term “racemic” refers to a mixture of equal parts of enantiomers and which mixture is optically inactive.

The term “absolute configuration” refers to the spatial arrangement of the atoms of a chiral molecular entity (or group) and its stereochemical description, e.g., R or S.

The stereochemical terms and conventions used in the specification are meant to be consistent with those described in Pure & Appl. Chem 68:2193 (1996), unless otherwise indicated.

The term “enantiomeric excess” or “ee” refers to a measure for how much of one enantiomer is present compared to the other. For a mixture of R and S enantiomers, the percent enantiomeric excess is defined as R−S|*100, where R and S are the respective mole or weight fractions of enantiomers in a mixture such that R+S=1. With knowledge of the optical rotation of a chiral substance, the percent enantiomeric excess is defined as ([α]_{obs}/[α]ₘₐₓ)*100, where [α]_{obs} is the optical rotation of the mixture of enantiomers and [α]ₘₐₓ is the optical rotation of the pure enantiomer. Determination of enantiomeric excess is possible using a variety of analytical techniques, including NMR spectroscopy, chiral column chromatography, or optical polarimetry.

The use of the terms “a”, “an”, “the”, and similar referents in the context of describing the disclosure (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

### Synthesis of Compounds of the Disclosure

The disclosure also provides the following embodiments directed to methods of making a Compound of the Disclosure.

Embodiment 1. A method of preparing compound 1:

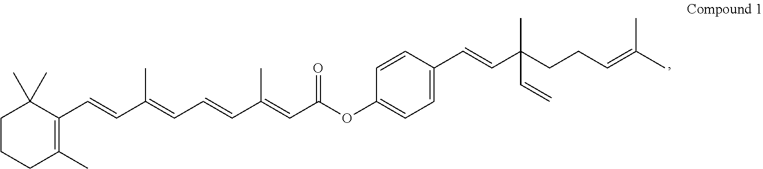 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of geometric isomers and/or stereoisomers thereof, the method comprising:

(a) reacting retinoic acid, or a geometric isomer thereof, with ClC(O)OR, wherein R is a C₁-C₁₀ alkyl group, in solvent to give a carbonic anhydride;

(b) reacting the carbonic anhydride of (a) with bakuchiol, or a geometric isomer and/or stereoisomer thereof, in a solvent to give Compound 1, or a geometric isomer and/or stereoisomer thereof; and, optionally,

(c) isolating Compound 1, or a geometric isomer and/or stereoisomer thereof.

Embodiment 2. The method of embodiment 1, wherein in R is methyl, ethyl, propyl, butyl, or isobutyl.

Embodiment 3. The method of Embodiment 1 or 2, wherein the molar ratio of ClC(O)OR to retinoic acid in (a) is from about 1:1 to about 1.1:1.

Embodiment 4. The method of any one of Embodiments 1-3, wherein (a) further comprises reacting the retinoic acid and ClC(O)OR in the presence of a base, e.g., trimethylamine, triethylamine, diisopropylethylamine, or pyridine.

Embodiment 5. The method of Embodiment 4, wherein the molar ratio of base to retinoic acid in (a) is from about 1:1 to about 5:1.

Embodiment 6. The method of any one of Embodiments 1-5, wherein the solvent in (a) is selected from the group consisting of anhydrous tetrahydrofuran, dichloromethane, dioxane, or tetrahydrofuran, or a mixture thereof.

Embodiment 7. The method of any one of Embodiments 1-6, wherein the reaction in (a) is performed at a temperature of from about 0° C. to about 25° C.

Embodiment 8. The method of any one of Embodiments 1-7, wherein the molar ratio of bakuchiol to carbonic anhydride of (a) in (b) is from about 0.75:1 to about 1.25:1.

Embodiment 9. The method of any one of Embodiments 1-8, wherein (b) further comprises reacting the carbonic anhydride of (a) and bakuchiol in the presence of a base, e.g., trimethylamine, triethylamine, diisopropylethylamine, pyridine, or 4-dimethylaminopyridine.

Embodiment 10. The method of any one of Embodiments 1-9, wherein the solvent in (b) is acetonitrile, dioxane, or tetrahydrofuran.

Embodiment 11. The method of any one of Embodiments 1-10, wherein the reaction in (b) is performed at a temperature of from about 0° C. to about 60° C.

Embodiment 12. The method of any one of Embodiments 1-11, wherein (b) further comprises reacting the carbonic anhydride of (a) and bakuchiol in the presence of ethanolamine.

Embodiment 13. The method of any one of Embodiments 1-12 according to Scheme 2:

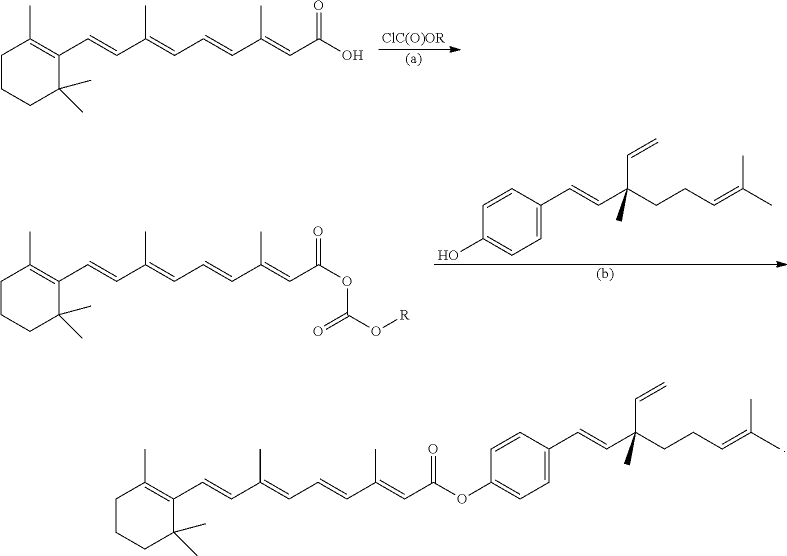

Embodiment 14. The method of Embodiment 13, wherein R is isobutyl.

Embodiment 15. The method of Embodiment 13, wherein R is ethyl.

Embodiment 16. The method of any one of Embodiments 13-15, wherein the solvent in (a) is tetrahydrofuran.

Embodiment 17. The method of any one of Embodiments 13-16, wherein (a) further comprises reacting the retinoic acid and ClC(O)OR in the presence of triethylamine.

Embodiment 18. The method of any one of Embodiments 13-17, wherein the solvent in (b) is acetonitrile.

Embodiment 19. The method of any one of Embodiments 13-18, wherein (b) further comprises reacting the carbonic anhydride of (a) and bakuchiol in the presence of ethanolamine.

### Compositions

In one embodiment, the disclosure provides a composition comprising a Compound of the Disclosure, or a geometric isomer and/or stereoisomer thereof, or a mixture thereof, and a pharmaceutically, dermatologically, or cosmetically acceptable carrier and/or excipient.

In another embodiment, the composition comprises a Compound of the Disclosure present at a concentration of from about 0.01% w/w to about 0.05% w/w, from about 0.010% w/w to about 0.10% w/w, from about 0.010% w/w to about 0.5% w/w, from about 0.01% w/w to about 1% w/w, from about 0.01% w/w to about 1.5% w/w, from about 0.01% w/w to about 2% w/w, from about 0.01% w/w to about 2.5% w/w, from about 0.01% w/w to about 3% w/w, from about 0.01% w/w to about 3.5% w/w, from about 0.01% w/w to about 4% w/w, from about 0.01% w/w to about 5% w/w, from about 0.01% w/w to about 6% w/w, from about 0.01% w/w to about 7% w/w, from about 0.01% w/w to about 8% w/w, from about 0.01% w/w to about 9% w/w, from about 0.01% w/w to about 10% w/w, from about 0.05% w/w to about 0.1% w/w, from about 0.05% w/w to about 0.5% w/w, from about 0.05% w/w to about 1% w/w, from about 0.05% w/w to about 1.5% w/w, from about 0.05% w/w to about 2% w/w, from about 0.05% w/w to about 2.5% w/w, from about 0.05% w/w to about 3% w/w, from about 0.05% w/w to about 3.5% w/w, from about 0.05% w/w to about 4% w/w, from about 0.05% w/w to about 5% w/w, from about 0.05% w/w to about 6% w/w, from about 0.05% w/w to about 7% w/w, from about 0.05% w/w to about 8% w/w, from about 0.05% w/w to about 9% w/w, from about 0.05% w/w to about 10% w/w, from about 0.10% w/w to about 0.5% w/w, from about 0.10% w/w to about 1% w/w, from about 0.10% w/w to about 1.50% w/w, from about 0.1% w/w to about 2% w/w, from about 0.1% w/w to about 2.5% w/w, from about 0.1% w/w to about 3% w/w, from about 0.1% w/w to about 3.5% w/w, from about 0.1% w/w to about 4% w/w, from about 0.1% w/w to about 5% w/w, from about 0.1% w/w to about 6% w/w, from about 0.1% w/w to about 7% w/w, from about 0.1% w/w to about 8% w/w, from about 0.1% w/w to about 9% w/w, from about 0.10% w/w to about 10% w/w, from about 0.50% w/w to about 10% w/w, from about 0.50% w/w to about 1.50% w/w, from about 0.50% w/w to about 2% w/w, from about 0.50% w/w to about 2.50% w/w, from about 0.50% w/w to about 30% w/w, from about 0.50% w/w to about 3.5% w/w, from about 0.5% w/w to about 4% w/w, from about 0.5% w/w to about 5% w/w, from about 0.5% w/w to about 6% w/w, from about 0.5% w/w to about 7% w/w, from about 0.5% w/w to about 80% w/w, from about 0.5% w/w to about 9% w/w, from about 0.5% w/w to about 2% w/w, from about 1% w/w to about 1.50% w/w, from about 1% w/w to about 2% w/w, from about 1% w/w to about 2.5% w/w, from about 1% w/w to about 3% w/w, from about 1% w/w to about 3.5% w/w, from about 1% w/w to about 4% w/w, from about 1% w/w to about 5% w/w, from about 1% w/w to about 6% w/w, from about 1% w/w to about 7% w/w, from about 1% w/w to about 8% w/w, from about 1% w/w to about 9% w/w, from about 1% w/w to about % w/w, from about 1.5% w/w to about 2% w/w, from about 1.5% w/w to about 2.5% w/w, from about 1.5% w/w to about 34 w/w, from about 1.5% w/w to about 3.5% w/w, from about 1.5% w/w to about 4% w/w, from about 1.5% w/w to about 5% w/w, from about 1.5% w/w to about 6% w/w, from about 1.5% w/w to about 7% w/w, from about 1.5% w/w to about 8% w/w, from about 1.5% w/w to about 9.% w/w, from about 1.5% w/w to about 3% w/w, from about 2% w/w to about 2.5% w/w, from about 2% w/w to about 3% w/w, from about 2% w/w to about 3.5% w/w, from about 2% w/w to about 4% w/w, from about 2% w/w to about 5% w/w, from about 2% w/w to about 6% w/w, from about 2% w/w to about 7% w/w, from about 2% w/w to about 8% w/w, from about 2.5% w/w to about 9% w/w, from about 2% w/w to about 10% w/w, from about 2.5% w/w to about 3% w/w, from about 2.5% to w/w to about 3.5% w/w, from about 2.5% w/w to about 4% w/w, from about 2.5% w/w to about 5% w/w, from about 2.5% w/w to about 6% w/w, from about 2.5% w/w to about 7% w/w, from about 2.5% w/w to about 8% w/w, from about 2.5% w/w to about 9% w/w, from about 2.3% w/w to about 4% w/w, from about 3% w/w to about 3.5% w/w, from about 3% w/w to about 4% w/w, from about 3% w/w to about 5% w/w, from about 3 to w/w to about 6% w/w, from about 3% w/w to about 7% w/w, from about 3% w/w to about 8% w/w, from about 3% w/w to about 9% w/w, from about 3% w/w to about 10% w/w, from about 3.5% w/w to about 4% w/w, from about 3.5% w/w to about 5% w/w, from about 3.5% w/w to about 6% w/w, from about 3.5% w/w to about 7% w/w, from about 3% w/w to about 8% w/w, from about 3.4% w/w to about 9% w/w, from about 3.5% w/w to about 6% w/w, from about 4% w/w to about 5% w/w, from about 4% w/w to about 6% w/w, from about 4% w/w to about 7% w/w, from about 4% w/w to about 8% w/w, from about 4% w/w to about 9% w/w, from about 4% w/w to about 10% w/w, from about 5% w/w to about 6% w/w, from about 5% w/w to about 7% w/w, from about 5% w/w to about 8% w/w, from about 5% w/w to about 9% w/w, from about 5% w/w to about 10% w/w, from about 6% w/w to about 7 w/w, from about 6% w/w to about 8% w/w, from about 6% w/w to about 9% w/w, from about 6% w/w to about 9% w/w, from about 7% w/w to about 8% w/w, from about 7% w/w to about 9% w/w, from about 7% w/w to about 10% w/w, from about 8% w/w to about 9% w/w, from about 8% w/w to about 10% w/w, or from about 9% w/w to about 10% w/w.

In another embodiment, the composition comprises a Compound of the Disclosure present at a concentration of about 1% w/w. In another embodiment, the composition comprises a Compound of the Disclosure present at a concentration of about 0.01% w/w, about 0.05% w/w, about 0.1% w/w, about 0.5% w/w, about 1.5% w/w, about 2% w/w, about 2.5% w/w, about 3% w/w, about 3.5% w/w, about 4% w/w, about 5% w/w, about 6% w/w, about 7% w/w, about 8% w/w, about 9% w/w, or about 10% w/w.

In another embodiment, the composition comprises a Compound of the Disclosure having a chemical purity of 90% or more as measured, for example, by high-performance liquid chromatography (HPLC). In another embodiment, the composition comprises a Compound of the Disclosure having a chemical purity of 95% or more, 97% or more, 98% or more, or 99% or more.

In another embodiment, the composition comprises a Compound of the Disclosure having an isomeric purity of 50% or more as measured, for example, by chiral HPLC. In another embodiment, the composition comprises a Compound of the Disclosure having an isomeric purity of 55% or more, 60% or more, 65% or more, 70% or more, 75% or more, 80% or more, 85% or more, 90% or more, 95% or more, 97% or more, 98% or more, or 99% or more.

In addition to a Compound of the Disclosure, the composition further comprises a pharmaceutically, dermatologically, or cosmetically acceptable carrier. The carrier can be in a wide variety of forms including, for example, liquids, lotions, creams, masks, toners, serums, gels, foams, emulsions, dispersions, sprays, liposomes, coacervates, ointments, or transdermal patches. The carrier may be an aqueous-based solution or cleanser; an alcohol-based solution or gel; an ointment based on fats, waxes, animal and vegetable oils, and solid and liquid hydrocarbons; or an emulsion carrier, including, but not limited to, oil-in-water, water-in-oil, water-in-oil-in-water, and oil-in-water-in-silicone emulsions. The carrier can also be formulated as alcohol or water based cleansers, toilet bars, liquid soaps, shampoos, bath gels, hair conditioners, hair tonics, pastes or mousses. The carrier will generally comprise from about 30% to about 99.99%, preferably from about 50% to about 99.9%, more preferably from about 80% to about 99%, most preferably from about 85% to about 95% of the skin treatment composition of the present invention based on the combined weight of the actives and the carrier.

In another embodiment, the composition further comprises one or more skin penetrants. Skin penetrants enhance and/or expedite the penetration of the Compound of the Disclosure through the skin layers to, in particular, the targeted basal keratinocytes, the dermal epidermal junction, extracellular matrix, and/or the tight junction. Skin penetrants are additives that, when applied to the skin, have a direct effect on the permeability of the skin barrier: increasing the speed with which and/or the amount by which certain other compounds are able to penetrate into the skin layers. Exemplary organic penetration enhancers include dimethyl sulfoxide (DMSO); isopropyl myristate; decyl, undecyl or dodecyl alcohol; propylene glycol; polyethylene glycol; C₉-C₁₁, C₁₂-C₁₃ or C₁₂-C₁₅ fatty alcohols; azone; alkyl pyrrolidones; diethoxy glycol (Transcutol); lecithin; etc. Surfactants can also be used as penetration enhancers.

In another embodiment, the composition further comprises a pH adjuster. pH adjusters include, but are not limited to, ammonia, sodium carbonate, sodium hydroxide, ammonium hydroxide, potassium hydroxide, arginine, triethanolamine, hydrochloric acid, phosphoric acid, sodium hydrogen phosphate, sodium dihydrogen phosphate, and citric acid.

In another embodiment, the pH of the composition is from about 4.5 to about 7.5. In another embodiment, the pH of the composition is from about 4.5 to about 5, from about 4.5 to about 5.5, from about 4.5 to about 6, from about 4.5 to about 6.5, from about 4.5 to about 7, from about 4.5 to about 7.5, from about 5 to about 5.5, from about 5 to about 6, from about 5 to about 6.5, from about 5 to about 7, from about 5 to about 7.5, from about 5.5 to about 6, from about 5.5 to about 6.5, from about 5.5 to about 7, from about 5.5 to about 7.5, from about 6 to about 6.5, from about 6 to about 7, from about 6 to about 7.5, from about 6.5 to about 7, from about 6.5 to about 7.5, or from about 7 to about 7.5. In another embodiment, the pH of the composition is about 7. In another embodiment, the pH of the composition is about 4.5, about 5, about 5.5, about 6, about 6.5, about 7, or about 7.5.

In another embodiment, the composition further comprises a viscosity modifier. Viscosity modifiers include, but are not limited to, water-soluble polyacrylic and hydrophobically modified polyacrylic resins such as Carbopol and Pemulen, starches such as corn starch, potato starch, and tapioca, gums such as guar gum, gum arabic, and xanthan gum, sclerotium gum, microcrystalline cellulose, and cellulose ethers such as hydroxypropyl cellulose, hydroxyethyl cellulose, and carboxymethyl cellulose.

In another embodiment, the composition further comprises an effective amount of one or more skin-protective or treatment ingredients, including antioxidants, sunscreen actives, skin lightening actives, exfoliants, anti-acne actives, vitamins, anti-inflammatory agents, self-tanning agents, moisturizers, emollients, humectants, compatible solutes, or combinations thereof.

In another embodiment, the composition further comprises hyaluronic acid. In another embodiment, the composition further comprises one or more ceremides. In another embodiment, the composition further comprises one or more collagen peptides.

In another embodiment, the composition further comprises one or more antioxidants. Suitable antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of the present disclosure include, but are not limited to, butylated hydroxytoluene, tocopherols (e.g., tocopherol acetate), tocotrienols, curcurmin and its derivatives and ubiquinone. Natural extracts containing antioxidants suitable for use in the compositions of the present disclosure include, but are not limited to, extracts containing flavonoids and isoflavonoids and their derivatives (e.g., genistein and diadzein), and extracts containing resveratrol. Examples of such natural extracts include grape seed, green tea, pine bark, Phyllanthus emblica and propolis.

In another embodiment, the composition further comprises one or more sunscreen actives. Sunscreen actives are of two types, inorganic actives that work by reflecting the UV light and organic actives that work, predominately, by absorbing UV energy. The amount of the sunscreen active to be incorporated into the sunscreen effective formulations is that which is conventional in the art. Typically, the amount is dependent upon, among other factors, the delivery means, e.g., applied as a spray or lotion; the stability of the active; the efficacy of the selected sunblock active itself; and the application rate, as well as the particular SPF desired.

Organic sunscreen actives include, but are not limited to, butyl methoxydibenzoylmethane (avobenzone), benzophenone-8, dioxybenzone, homosalate, octylsalate, menthyl anthranilate, octocrylene, ethyhexyl methoxycinnamate (Octinoxate), oxybenzone, ethylhexyl salicylate (Octisalate), benzophenone-3, ethylhexyl dimethyl PABA (Padimate O), glyceryl PABA, phenylbenzimidazole sulfonic acid, sulfisobezone, trolamine salicylate, 4-methylbenzylidene camphor, bisoctriazole, bemotrizinol, ecamsule, drometrizole trisiloxane, disodium phenyl dibenzimidazole tetrasulfonate, diethylamine hydroxybenzoyl hexyl benzoate, octyl triazone, hexyl benzoate, benzophenone-4, ethyhexyl triazone, diethylhexyl butamido triazone, bisimidazylate, and polysilicone-15.

Inorganic sunscreens include, but are not limited to, microfine surface treated titanium dioxide and microfine untreated and surface treated zinc oxide. The titanium dioxide in the sunscreen compositions preferably has a mean primary particle size of between 5 and 150 nm, preferably between 10 and 100 nm. Titanium oxide may have an anatase, rutile, or amorphous structure. The zinc oxide in the sunscreen compositions preferably has a mean primary particle size of between 5 nm and 150 nm, preferably between 10 nm and 100 nm. Examples of suitable hydrophobically modified titanium dioxide compositions include, but are not limited to, UV Titans® X161, M160, M262 (surface treated with stearic acid and alumina); Eusolex® T-2000 (surface treated with alumina and simethicone); T-Cote® (surface treated with dimethicone); Mirasun® TiW60 (surface treated with silica and alumina); Tayaca MT100T (surface treated with aluminum stearate); Tayaca MT-100SA (surface treated with silica and alumina); Tayaca MT-500SA (surface treated with silica and alumina); Tioveile EUT, FIN, FLO, FPT, GCM, GPT, IPM, MOTG, OP, TG, TGOP (surface treated with silica and alumina, 40% dispersion in a range of cosmetic vehicle); Eusolexe T-45D (surface treated with alumina and simethicone, 45% dispersion in isononoyinonaoate); and Eusolex® T-Aqua (surface treated with aluminum hydroxide, 25% dispersion in water). Examples of suitable untreated and hydrophobically modified zinc oxide include but are not limited to: Z-Cote® (uncoated microfine zinc oxide); Z-Cote® HP-1 (surface treated with dimethicone); Sachtotec® LA 10 (surface treated with lauric acid); Sachtotec® (uncoated microfine zinc oxide); Spectraveil® FIN, IPM, MOTG, OP, TG, TGOP (uncoated, 60% dispersion in a range of cosmetic vehicle); Z-sperse® TN (untreated, dispersion in C12-15 alkyl benzoate); and Z-sperse® TN (untreated, dispersion in octydodecyl neopentanoate).

In another embodiment, the composition further comprises one or more skin lightening actives. Skin lightening actives can decrease the amount of melanin in the skin or provide such an effect by other mechanisms. Skin lightening actives include, but are not limited to, adapalene, aloe extract, alpha-glycaryl-L-ascorbic acid, aminotyroxine, ammonium lactate, anethole derivatives, apple extract, arbutin, Areca catechu L. extract, ascorbic acid, ascorbyl palmitate, azelaic acid, bamboo extract, bearberry extract, Bletilla tuber, Bupleurum falcatum extract, burnet extract, Burnet Power (available from Barnet Products), butyl hydroxy anisole, butyl hydroxy toluene, butyl resoreinol, Chuanxiong, Cola decaballo extract, Dang-Gui, deoxyarbutin, 1,3 diphenyl propane derivatives, 2,5 dihydroxybenzoic acid and its derivatives, 2-(4-acetoxyphenyl)-1,3 dithane, 2-(4-hydroxyphenyl)-1,3 dithane, ellagic acid, escinol, estragole derivatives, esculoside, esculetin, FADEOUT (available from Pentapharm), Fangfeng, fennel extract, gallic acid and its derivatives, ganodenna extract, gaoben, GATULINE WHITENING (available from Gattlefosse), genistic acid and its derivatives, gentisyl alcohol, glabridin and its derivatives, gluco pyranosyl-1-ascorbate, gluconic acid, glucosamine, glycolic acid, glycyrrhizinic acid, green tea extract, 4-Hydroxy-5-methyl-3[2H]-furanone, hydroquinine, 4 hydroxyanisole and its derivatives, 4-hydroxy benzoic acid derivatives, hydroxycaprylic acid, hyptis extract, inositol ascorbate, kojic acid, kojic dipalnitate, lactic acid, lemon extract, licorice extract, Licorice P-TH (available from Barnet Products), linoleic acid, Melfade (available from Pentapharm), MELAWHITE (available from Pentapharm), Melanostatine DM (available from Laboratories Seporga), Morus alba extract, mulberry root extract, niacinamide, 5-octanoyl salicylic acid, parsley extract, Phellinus linteus extract, Pinon blanco extract, Pinon negro extract, piri-piri extract, pyrogallol derivatives, retinoic acid, retinol, retinyl esters (acetate, propionate, palmitate, linoleate), 2,4 resorcinol derivatives, 3,5 resorcinol derivatives, rose fruit extract, rucinol, salicylic acid, Song-Yi extract, Sophora Powder (available from Barnet Products), 4-thioresorein, 3,4,5 trihydroxybenzyl derivatives, tranexamic acid, tyrostat (Rumex Extract available from Fytokem), Tyroslat 10,11 (available from Fytokem), vanilla derivatives, vitamin D3 and its analogs, and mixtures thereof.

In another embodiment, the composition further comprises one or more exfoliants. Exfoliants include, but are not limited to, alpha-hydroxy acids such as lactic acid, glycolic acid, malic acid, tartaric acid, citric acid, or any combination of any of the foregoing, beta-hydroxy acids such as salicylic acid, polyhydroxy acids such as lactobionic acid and gluconic acid, and mechanical exfoliation such as microdermabrasion.

In another embodiment, the composition further comprises one or more anti-acne actives. Anti-acne actives include, but are not limited to, the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, a preferred example of which is N-acetyl-L-cysteine; lipoic acid; antibiotics and antimicrobials such as benzoyl peroxide, octopirox, tetracycline, 2,4,4′-trichloro-2′-hydroxy diphenyl ether, 3,4,4′-trichlorobanilide, azelaic acid and its derivatives, phenoxyethanol, phenoxypropanol, phenoxyisopropanol, ethyl acetate, clindamycin and meclocycline; sebostats such as flavonoids and bioflavonoids; bile salts such as scymnol sulfate and its derivatives, deoxycholate, and cholate; abietic acid; adapalene; allantoin; aloe extracts; arbietic acid and its salts; aryl-2,4 dioxo oxazolidine derivatives; ASEBIOL (available from Laboratories Serobiologiques, located in Somerville, N.J.); azaleic acid; barberry extracts; bearberry extracts; Belamcanda chinensis; benzoquinolinones; benzoyl peroxide; berberine; BIODERMINE (available from Sederma, located in Brooklyn, N.Y.); bioflavinoids; bisabolol; S-carboxymethyl cysteine; carrot extracts; cassin oil; clove extracts; citral; citronellal; climazole; Completech MBAC-OS (available from Lipo); CREMOGEN M82 (available from Dragoco, located in Totowa, N.J.); cucumber extracts; dehydroacetic acid and its salts; dehydroeplandersterone salicylate; dichlorophenyl imidazoldioxolan which is commercially available as COMPLETECH MBAC-OS (from Lipo, located in Paterson, N.J.); DL valine and its esters; DMDM hydantoin; Epicutin TT (available from CLR); erythromycin; escinol; ethyl hexyl monoglyceryl ether; ethyl 2-hydroxy undecanoate; farnesol; farnesol acetate; geranoil; glabridin; gluconic acid; gluconolactone; glyceryl monocaprate; glycolic acid; grapefruit seed extract; gugu lipid; Hederagenin (available from Maruzen); hesperitin; hinokitol; hops extract; hydrogenated rosin; 10 hydroxy decanoic acid; ichtyhol; interleukin 1 alpha antagonists; iodo-2-propynyl butyl carbamate; Kapilarine (available from Greentech); ketoconazole; lactic acid; lemon grass oil; Lichochalcone LR15 (available from Maruzen); linoleic acid; LIPACIDE C8CO (available from Seppic, located in Paris, France); lovastatin; 4 methoxysalicylic acid; metronidazole; minocycline; mukurossi; neem seed oil; vitamin B3 compounds (such as niacinamide and nicotinic acid); nisin; 5-octanoly salicylic acid; octopirox; panthenol; 1-pentadecanol; peonia extract; peppermint extract; phelladendron extract; 2-phenyl-benzothiophene derivatives; phloretin; PHLOROGINE (available from Secma); phosphatidyl choline; proteolytic enzymes; quercetin; red sandalwood extract; resorcinol; rosemary extract; rutin; sage extract; salicin; salicylic acid; skull cap extract; siber hegner extract; siberian saxifrage extract; silicol; sodium lauryl sulfate; sodium sulfoacetamide; Sophora Extract (available from Maruzen); sorbic acid; sulfur; sunder vati extract; tea tree oil; tetracyline; tetra hydroabietic acid; thyme extract; tioxolone; tocopherol; trehalose 6-undecylenoate; 3 tridecene-2-ol; triclosan; tropolone; UNITRIENOL T27 (available from Unichem, located in Gouda, Netherlands); vitamin D3 and its analogs; white thyme oil; willow bark extract; wogonin; Ylang Ylang; zinc glycerolate; zinc linoleate; zinc oxide; zinc pyrithione; zinc sulfate and mixtures thereof.

In another embodiment, the composition further comprises one or more vitamins. Vitamins and vitamin derivatives include, but are not limited to, vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C and derivatives (for example ascorbyl palmitate, ascorbyl glucoside, and ascorbyl acetate), vitamin D, ergocalciferol (vitamin D₂), vitamin E, DL-α-tocopherol, tocopherol E acetate, tocopherol hydrogensuccinate, vitamin Ki, esculin (vitamin P active ingredient), thiamine (vitamin B₁), nicotinic acid (niacin), niacinamide, pyridoxine, pyridoxal, pyridoxamine, (vitamin B₆), pantothenic acid, biotin, folic acid and cobalamine (vitamin B₁₂). In one embodiment, the vitamins are vitamin A palmitate, vitamin C and derivatives thereof, DL-α-tocopherol, tocopherol acetate, nicotinic acid, pantothenic acid and biotin.

In another embodiment, the composition further comprises one or more anti-inflammatory agents. Anti-inflammatory ingredients include, but are not limited to, bisabolol, curcurmin and its derivatives, retinoids, flavonoids, terpenes and other polyphenolics, as well as extracts and materials derived from Phellodendron amurense Cortex Extract (PCE), Non-Denatured Soy (Glycine max), Feverfew (Tanacetum parthenium), Ginger (Zingiber officinale), Ginko (Ginkgo biloba), Madecassoside (Centella asiatica extract ingredient), Cotinus (Cotinus coggygria), Butterbur Extract (Petasites hybridus), Goji Berry (Lycium barbarum), Milk Thistle Extract (Silybum marianum), Honeysuckle (Lonicera japonica), Basalm of Peru (Myroxylon pereirae), Sage (Salvia officinalis), Cranberry Extract (Vaccinium oxycoccos), Amaranth Oil (Amaranthus cruentus), Pomegranate (Punica granatum), Yerbe Mate (Ilex paraguariensis Leaf Extract), White Lily Flower Extract (Lilium candidum), Olive Leaf Extract (Olea europaea), Phloretin (apple extract), Oat Flour (Aveena sativa), Lifenol (Hops: Humulus lupulus) Extract, Bugrane P (Ononis spinosa), Licochalcone (Licorice: Glycyrrhiza inflate extract ingredient), Symrelief (Bisabolol and Ginger extract), and combinations thereof.

In another embodiment, the composition further comprises one or more self-tanning agents. Self-tanning agents include, but are not limited to, dihydroxyacetaone, tyrosine, tyrosine esters such as ethyl tyrosinate and glucose tyrosinate, acetyl tyrosine, phospho-DOPA, brazilin, caffeine, coffee extracts, dihydroxyacetone, DNA fragments, isobutyl methyl xanthine, methyl xanthine, Phototan (available from Laboratoires Serobiologiques), prostaglandins, tea extracts, theophylline, tyrosine, UNIPERTAN P2002 and UNIPERTAN P27 (both available from Unichem), and mixtures thereof.

In another embodiment, the composition further comprises one or more moisturizers. Moisturizers include, but are not limited to, C₁-C₂₀ alkyl esters of fatty acids, C₁₀-C₂₂ fatty acids (i.e., stearyl, palmityl, lauryl, myristyl acids), C₁₀-C₂₂ fatty alcohols (stearyl, palmityl, lauryl, myristyl, oleyl alcohols), and C₁₀-C₂₂ fatty alcohol ethers, C₁₆-C₂₂ alkanoic triglycerides (e.g., sunflower seed oil), sterols such as cholesterol and soy sterol, silicones (e.g., dimethicone), petroleum jelly, and mineral oils.

In another embodiment, the composition further comprises one or more emollients. Suitable emollients include those agents known for softening the skin which may be selected from hydrocarbons, fatty acids, fatty alcohols and esters. Petrolatum is a common hydrocarbon type of emollient conditioning agent. Other agents that may be employed include alkyl benzoate, mineral oil, polyolefins such as polydecene, and paraffins, such as isohexadecane. Fatty acids and alcohols used typically have from about 10 to 30 carbon atoms. Oily ester emollients may be those selected from one or more of the following: triglyceride esters, acetoglyceride esters, ethoxylated glycerides, alkyl esters of fatty acids, ether esters, polyhydric alcohol esters and wax esters. Additional emollients or hydrophobic agents include C₁₂-C₁₅ alkyl benzoate, dioctyladipate, octyl stearate, octyidodecanol, hexyl laurate, octyldodecyl neopentanoate, cyclomethicone, dicapryl ether, dimethicone, phenyl trimethicone, isopropyl myristate, capriylic/capric triglycerides, propylene glycol dicaprylate/dicaprate and decyl oleate, cyclomethicones and other silicone derivatives. Additional emollients include cetearyl alcohol, isoamyl laurate, glyceryl stearate citrate, glyceryl caprylate, caprylic/capric triglyceride, and cetearyl isononanoate.

In another embodiment, the composition further comprises one or more humectants. Humectants include, but are not limited to, various polyhydric alcohols, especially polyalkylene glycols and, more preferably, alkylene polyols and their derivatives. Exemplary humectants include, but are not limited to, propylene glycol, dipropylene glycol, polypropylene glycol, polyethylene glycol, sorbitol, 2-pyrrolidone-5-carboxylate, hydroxypropyl sorbitol, hexylene glycol, ethoxydiglycol 1,3-butylene glycol, 1,2,6-hexanetriol, glycerin, ethoxylated glycerin, propoxylated glycerin, and mixtures thereof.

In another embodiment, the composition further comprises one or more compatible solutes. Compatible solutes include, but are not limited to, ectoin, hydroxyectoin, taurines, carnitine, acetyl carnitine, and mixtures thereof.

In another embodiment, the composition further comprises one or more chelating agents. Chelating agents include, but are not limited to, disodium edetate (EDTA).

In another embodiment, the composition further comprises one or more functional actives. Functional actives include, but are not limited to, retinol (vitamin A), retinal, retinoic Acid, retinyl Propionate, retinyl Palmitate, retinyl Retinoate, sodium retinoyl hyaluronate (HyRetin®), Granactive Retinoid® (formulated with dimethyl isosorbide and hydroxypinacolone retinoate), AlphaRet®(ethyl lactyl retinoate), hyaluronic acid, Liposome CoQ10, niacinamide (B3), palmitoyl tripeptide-1, palmitoyl tripeptide-5, palmitoyl tetrapeptide-7, palmitoyl pentapeptide-4, palmitoyl pentapeptide-1, tetrapeptide 21, tetrapeptide 30, copper tripeptide-1, acetyl tetrapeptide-3, acetyl tetrapeptide-8, sh-oligopeptide-1(EGF), pentapetide-18, SYN-AKE (snake tripeptide), ceramide 1, ceramide 3 and 3b, ceramide 3 EP MB, ceramide NP, ceramide EOP, ceramide AP, ceramide 6-II, bakuchiol, vitamin E, panthenol (pro-vitamin of B5), allantoin, superoxide dismutase, resveratrol, ferulic acid, alguronic acid (usually added from algae extracts), and squalane (usually from olive oil).

In another embodiment, the composition further comprises glycerin. In another embodiment, the composition further comprises 1,3-propanediol.

In another embodiment, the composition further comprises a preservative. Preservatives include, but are not limited to, gluconolactone, sodium benzoate, phenoxyethanol, and Versatyl PC®(phenoxyethanol and caprylyl glycol).

In another embodiment, the composition is formulated for topical administration. In another embodiment, the composition is formulated for transdermal administration.

In another embodiment, the composition is formulated as an eye cream or serum. In another embodiment, the composition is formulated as an anti-wrinkle cream or serum. In another embodiment, the composition is formulated as a moisturizing cream. In another embodiment, the composition is formulated as a face wash. In another embodiment, the composition is formulated as a face wash. In another embodiment, the composition is formulated as a cosmetic.

In another embodiment, the composition is compatible with the skin microbiome.

In another embodiment, the composition does not cause longitudinal skin sensitivity when used continuously.

Methods of Use

In another embodiment, the disclosure provides a method of improving the common signs of cutaneous facial ageing in a subject, the method comprising administering to the subject an effective amount of a Compound of the Disclosure, or a composition thereof. In another embodiment, the common signs of cutaneous facial ageing are loss of elasticity, development of wrinkles and/or lines, increased pore size, cracking, flaking, uneven pigmentation, textural irregularities, or dyspigmentation, or a combination thereof.

In another embodiment, the disclosure provides a method of promoting or maintaining skin health and appearance in a subject, the method comprising administering to the subject an effective amount of a Compound of the Disclosure, or a composition thereof.

In another embodiment, the disclosure provides a method of preventing, ameliorating, or reducing the loss of softness and elasticity in the skin of a subject, the method comprising administering to the subject an effective amount of a Compound of the Disclosure, or a composition thereof.

In another embodiment, the disclosure provides a method of preventing, ameliorating, or reducing the presence of fine lines and/or wrinkles in the skin of a subject, the method comprising administering to the subject an effective amount of a Compound of the Disclosure, or a composition thereof.

In another embodiment, the disclosure provides a method of preventing, ameliorating, or reducing the presence of dark spots in the skin of a subject, the method comprising administering to the subject an effective amount of a Compound of the Disclosure, or a composition thereof.

In another embodiment, the disclosure provides a method of promoting or maintaining collagen production in the skin of a subject, the method comprising administering to the subject an effective amount of a Compound of the Disclosure, or a composition thereof.

In another embodiment, the disclosure provides a method of regulating Cutibacterium acnes in a subject, the method comprising administering to the subject and effective amount of a Compound of the Disclosure, or a composition thereof.

The disclosure also provides the following particular embodiments relating to methods and uses comprising a Compound of the Disclosure.

Embodiment I. A method of improving the common signs of cutaneous facial ageing in a subject, the method comprising administering to the subject an effective amount of a compound having the structure:

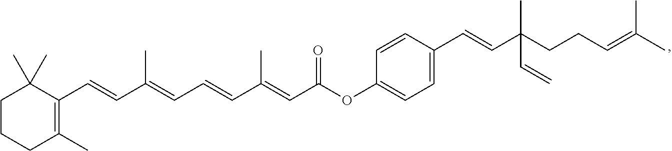 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment II. A method of promoting or maintaining skin health and appearance in a subject, the method comprising administering to the subject an effective amount of a compound having the structure:

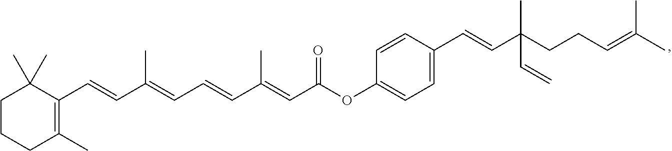 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment III. A method of preventing, ameliorating, or reducing the loss of softness and elasticity in the skin of a subject, the method comprising administering to the subject an effective amount of a compound having the structure:

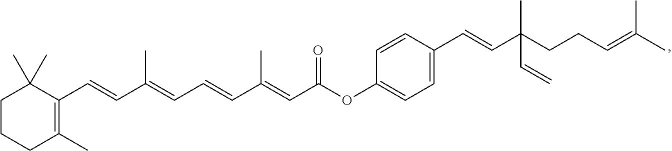 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment IV. A method of preventing, ameliorating, or reducing the presence of fine lines and/or wrinkles in the skin of a subject, the method comprising administering to the subject an effective amount of a compound having the structure:

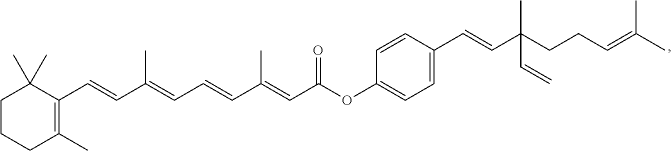 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment V. A method of preventing, ameliorating, or reducing the presence of dark spots in the skin of a subject, the method comprising administering to the subject an effective amount of a compound having the structure:

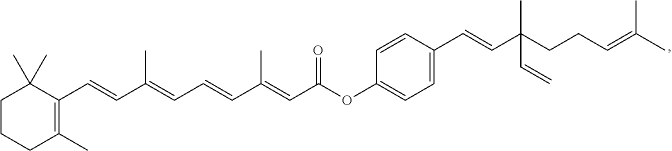 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment VI. A method of promoting or maintaining collagen production in the skin of a subject, the method comprising administering to the subject an effective amount of a compound having the structure:

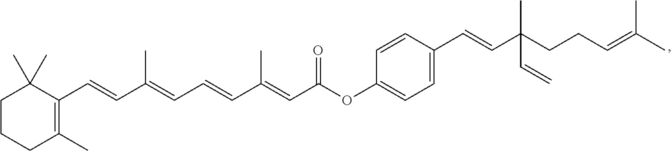 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment VII. The method of any one of Embodiments I-VI, wherein the compound is:

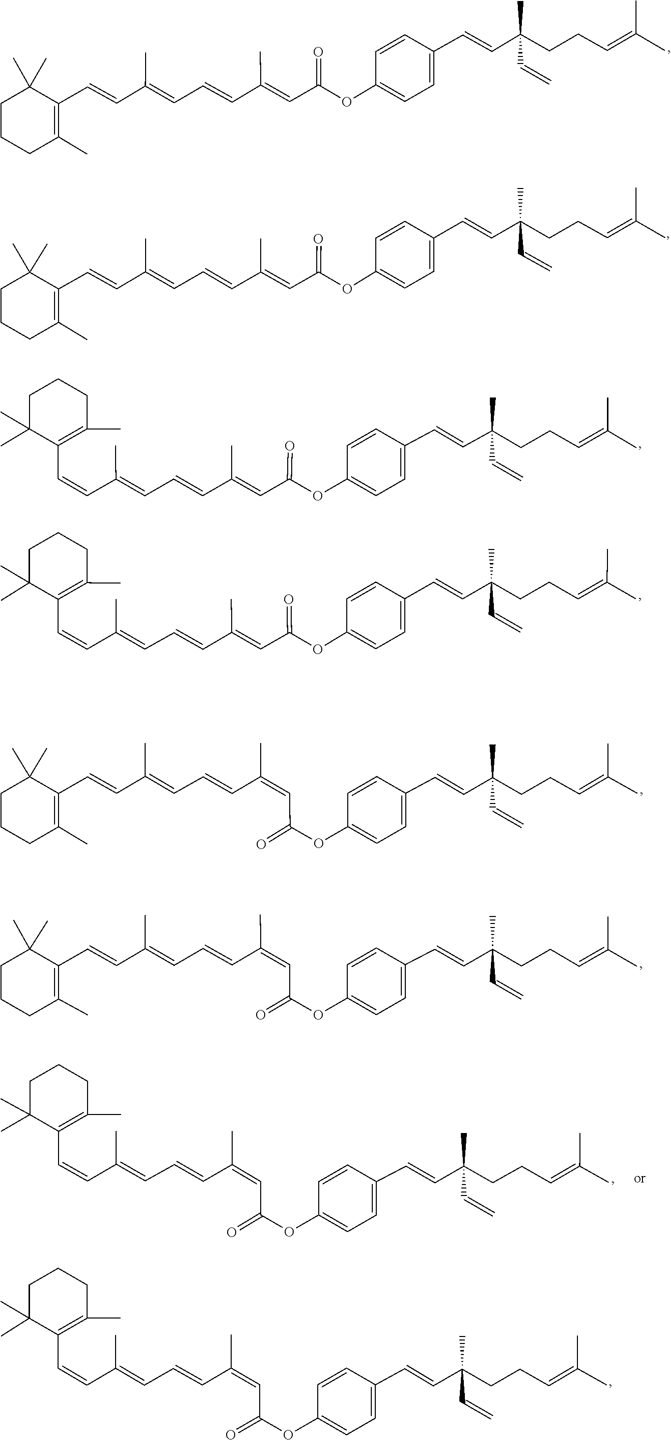

or a mixture thereof.

Embodiment VIII. The method of any one of Embodiments I-VII, wherein the compound is:

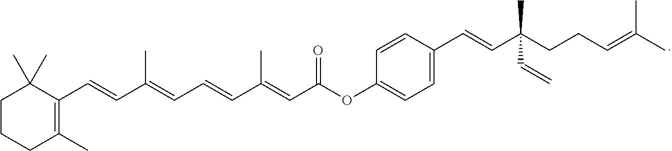

Embodiment IX. A compound having the structure:

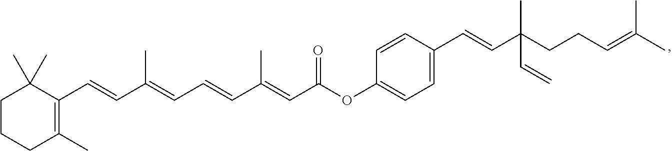 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric and/or stereoisomer thereof, or a mixture of isomers thereof, for use in improving the common signs of cutaneous facial ageing in a subject.

Embodiment X. A compound having the structure:

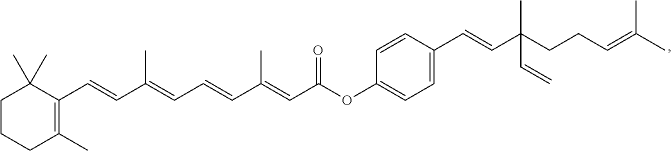 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric and/or stereoisomer thereof, or a mixture of isomers thereof, for use in promoting or maintaining skin health and appearance in a subject.

Embodiment XI. A compound having the structure:

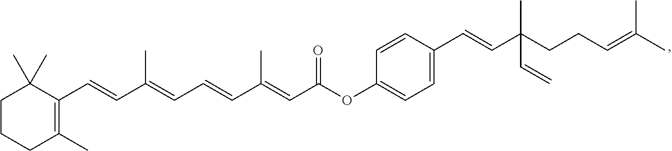 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric and/or stereoisomer thereof, or a mixture of isomers thereof, for use in preventing, ameliorating, or reducing the loss of softness and elasticity in the skin of a subject.

Embodiment XII. A compound having the structure:

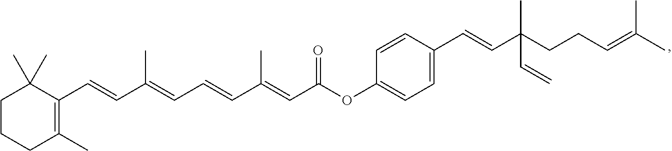 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric and/or stereoisomer thereof, or a mixture of isomers thereof, for use in preventing, ameliorating, or reducing the presence of fine lines and/or wrinkles in the skin of a subject.

Embodiment XIII. A compound having the structure:

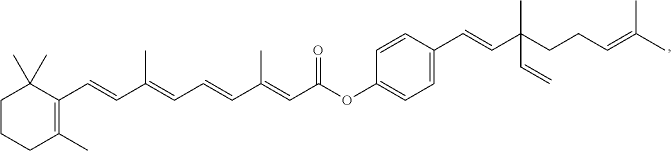 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric and/or stereoisomer thereof, or a mixture of isomers thereof, for use in preventing, ameliorating, or reducing the presence of dark spots in the skin of a subject.

Embodiment XIV. A compound having the structure:

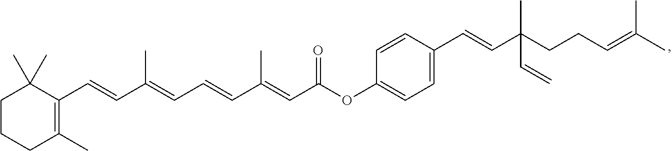 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric and/or stereoisomer thereof, or a mixture of isomers thereof, for use in promoting or maintaining collagen production in the skin of a subject.

Embodiment XV. The compound for use of any one of Embodiments IX-XIV, wherein the compound is:

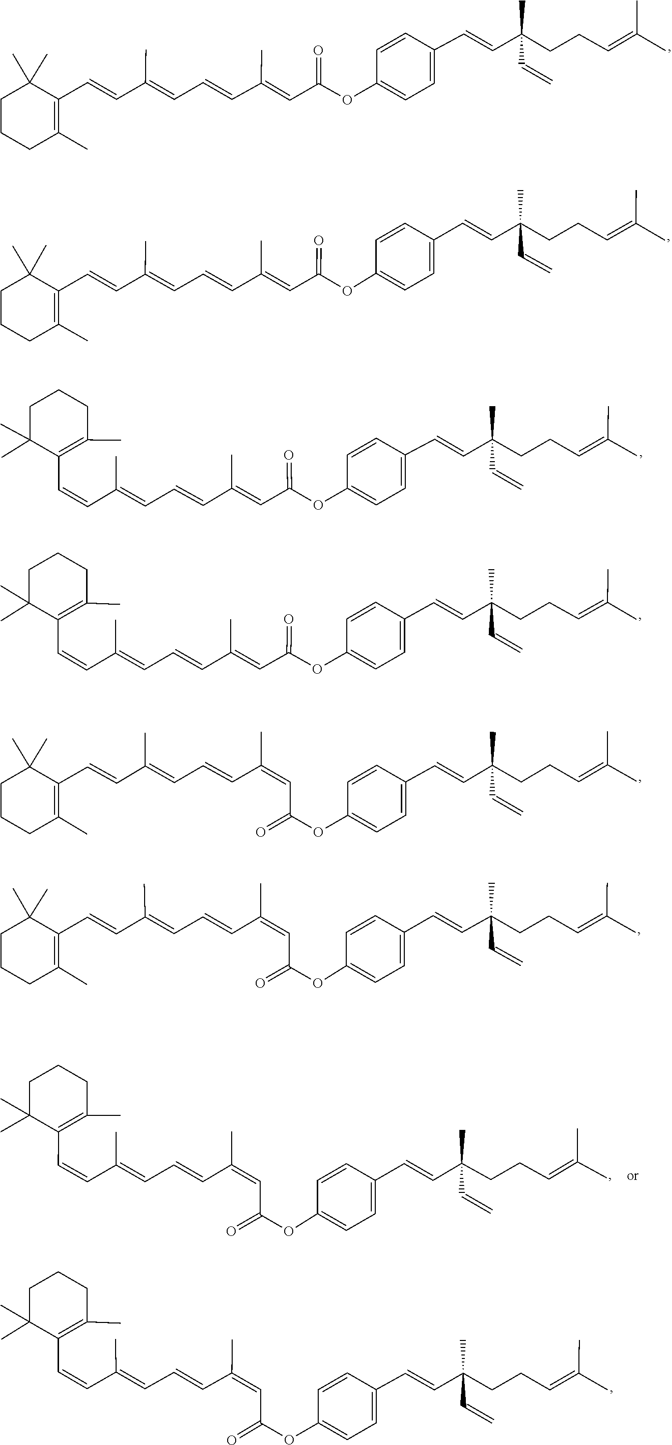

or a mixture thereof.

Embodiment XVI. The compound for use of any one of Embodiments IX-XV, wherein the compound is:

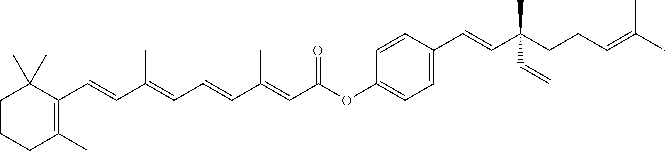

Embodiment XVII. A compound having the structure:

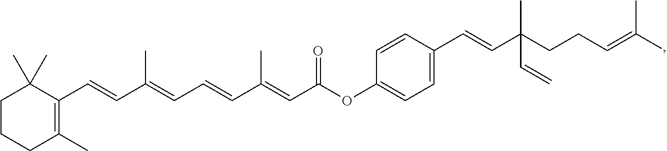

or a geometric and/or stereoisomer thereof, or a mixture of isomers thereof, for use in manufacture of a medicament for improving the common signs of cutaneous facial ageing in a subject.

Embodiment XVIII. A compound having the structure:

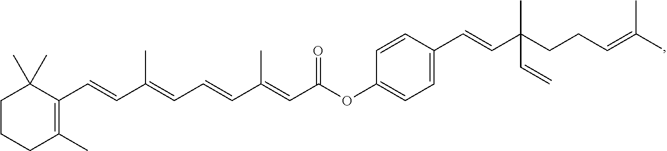

or a geometric and/or stereoisomer thereof, or a mixture of isomers thereof, for use in manufacture of a medicament for promoting or maintaining skin health and appearance in a subject.

Embodiment XIX. A compound having the structure:

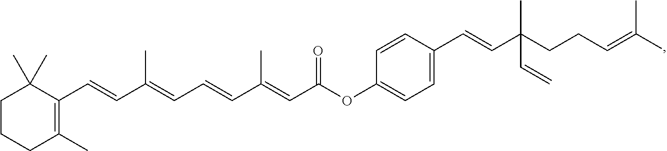

or a geometric and/or stereoisomer thereof, or a mixture of isomers thereof, for use in manufacture of a medicament for preventing, ameliorating, or reducing the loss of softness and elasticity in the skin of a subject.

Embodiment XX. A compound having the structure:

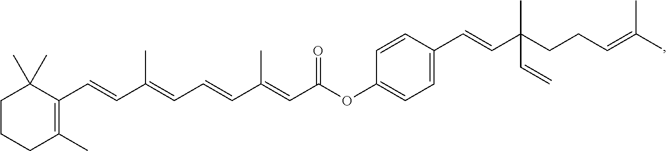

or a geometric and/or stereoisomer thereof, or a mixture of isomers thereof, for use in manufacture of a medicament for preventing, ameliorating, or reducing the presence of fine lines and/or wrinkles in the skin of a subject.

Embodiment XXI. A compound having the structure:

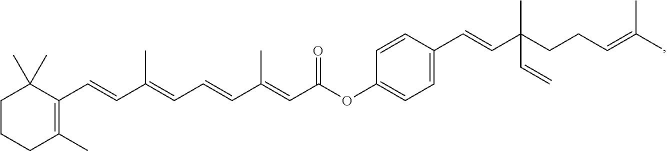

or a geometric and/or stereoisomer thereof, or a mixture of isomers thereof, for use in manufacture of a medicament for preventing, ameliorating, or reducing the presence of dark spots in the skin of a subject.

Embodiment XXII. A compound having the structure:

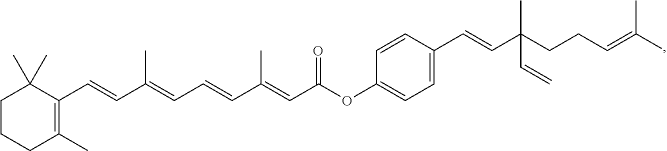

or a geometric and/or stereoisomer thereof, or a mixture of isomers thereof, for use in manufacture of a medicament for promoting or maintaining collagen production in the skin of a subject.

Embodiment XXIII. The use of any one of Embodiments XVII-XXII, wherein the compound is:

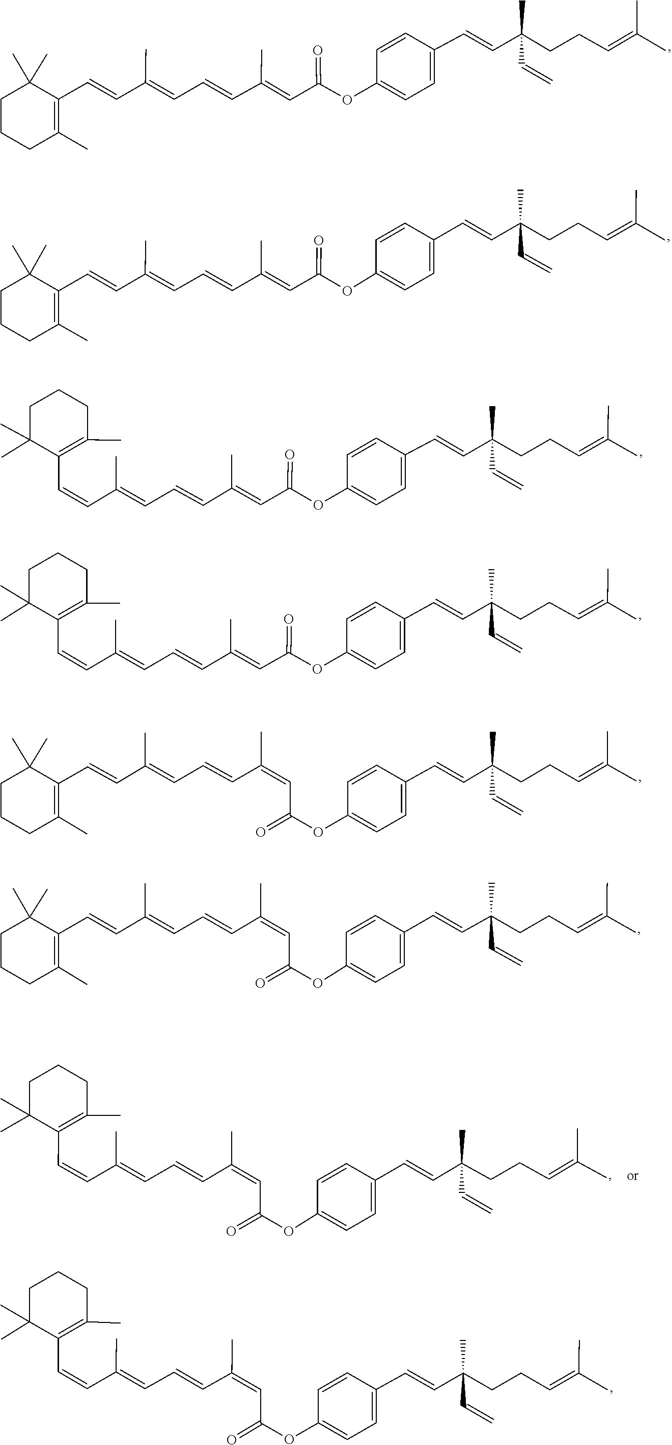

or a mixture thereof.

Embodiment XXIV. The use of any one of Embodiments XVII-XXIII, wherein the compound is:

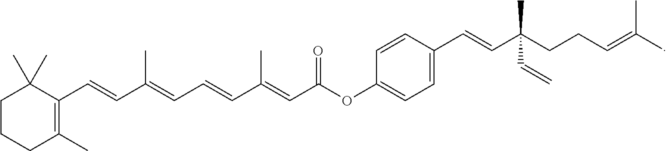

Embodiment XXV. A composition comprising a compound having the structure:

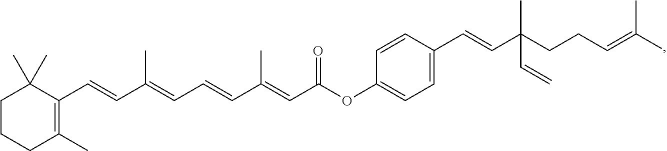

or a geometric and/or stereoisomer thereof, or a mixture of isomers thereof, for use in a method of improving the common signs of cutaneous facial ageing in a subject.

Embodiment XXVI. A composition comprising a compound having the structure:

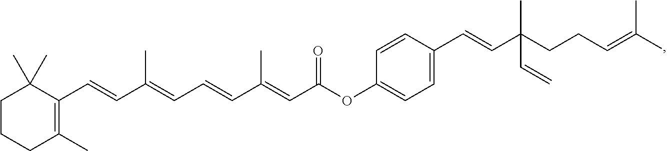

or a geometric and/or stereoisomer thereof, or a mixture of isomers thereof, for use in a method of promoting or maintaining skin health and appearance in a subject.

Embodiment XXVII. A composition comprising a compound having the structure:

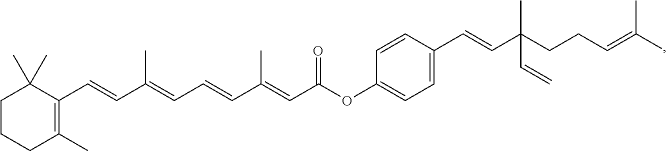

or a geometric and/or stereoisomer thereof, or a mixture of isomers thereof, for use in a method of preventing, ameliorating, or reducing the loss of softness and elasticity in the skin of a subject.

Embodiment XXVIII. A composition comprising a compound having the structure:

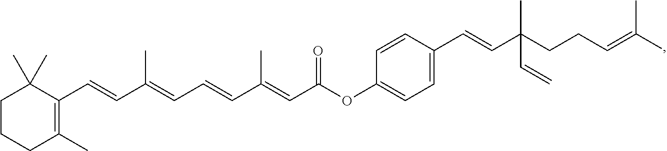

or a geometric and/or stereoisomer thereof, or a mixture of isomers thereof, for use in a method of preventing, ameliorating, or reducing the presence of fine lines and/or wrinkles in the skin of a subject.

Embodiment XXIX. A composition comprising a compound having the structure:

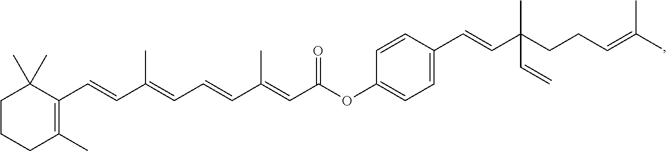

or a geometric and/or stereoisomer thereof, or a mixture of isomers thereof, for use in a method of preventing, ameliorating, or reducing the presence of dark spots in the skin of a subject.

Embodiment XXX. A composition comprising a compound having the structure:

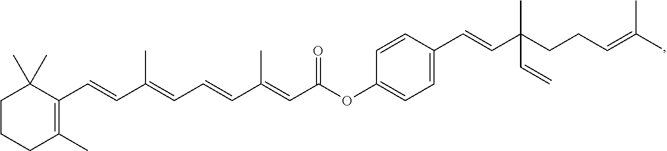

or a geometric and/or stereoisomer thereof, or a mixture of isomers thereof, for use in a method of promoting or maintaining collagen production in the skin of a subject.

Embodiment XXXI. The composition of any one of Embodiments XXV-XXX, wherein the compound is:

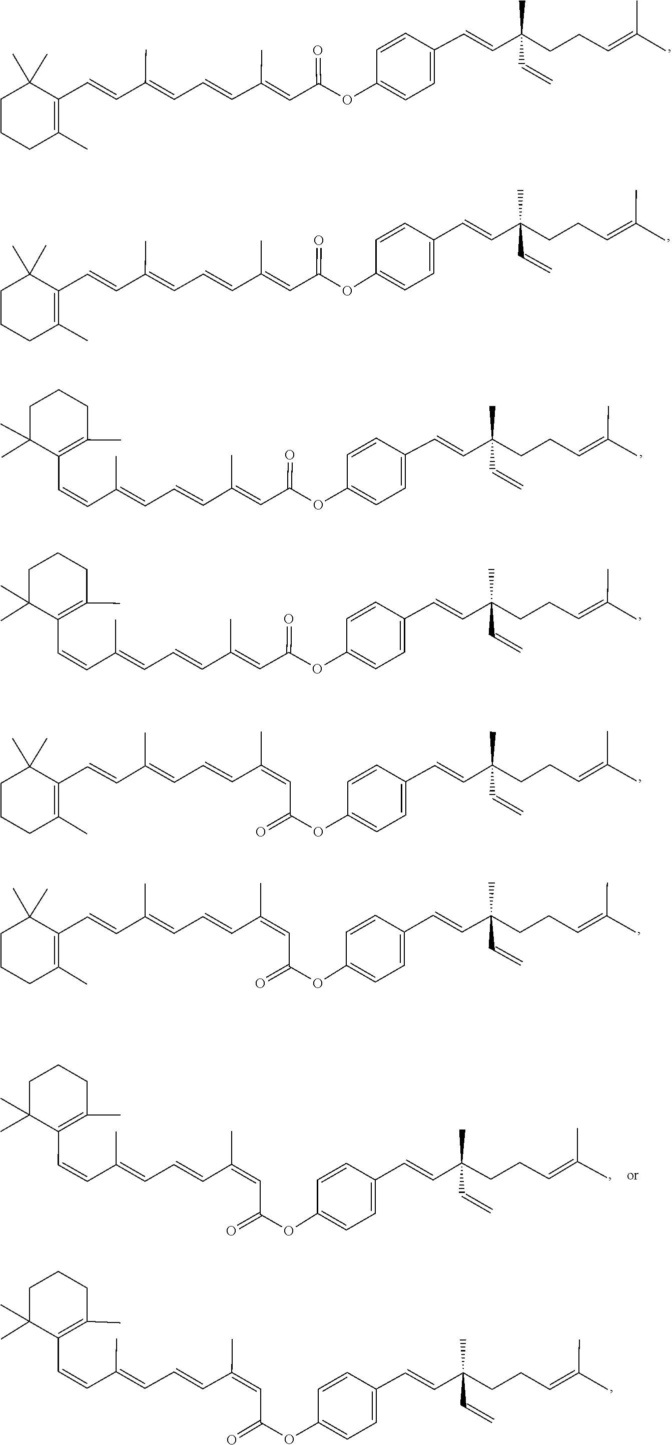

or a mixture thereof.

Embodiment XXXII. The composition of any one of Embodiments XXV-XXXI, wherein the compound is:

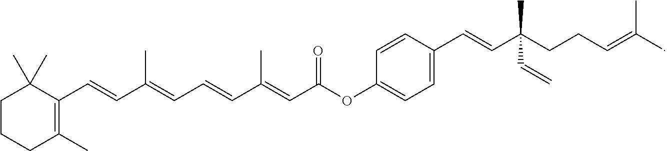

Embodiment XXXII. A method of administering an effective amount of bakuchiol and/or retinoic acid to a subject, the method comprising administering to the subject a compound having the structure:

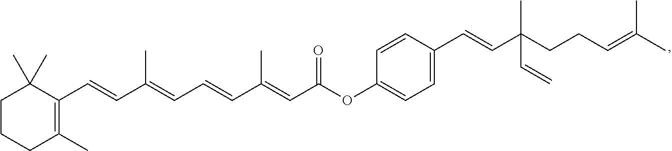 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment XXXIII. A method of mitigating longitudinal skin sensitivity caused by the continuous administration of retinol or retinoic acid to a subject, the method comprising administering to the subject an effective amount of a compound having the structure:

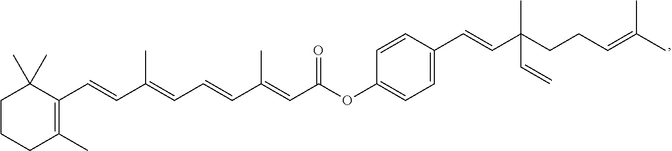 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof.

Embodiment XXXIV. The method of Embodiment XXXII or XXXIII, wherein the compound is:

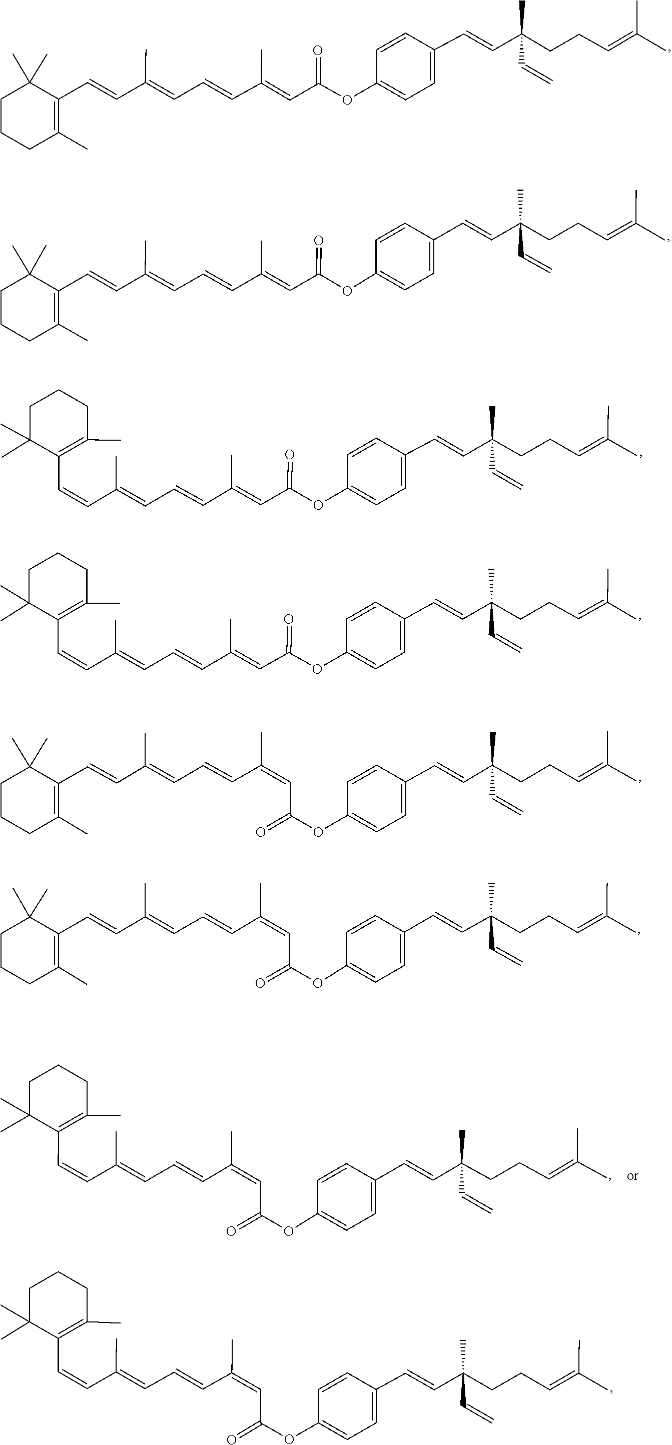

or a mixture thereof.

Embodiment XXXV. The method of any one of Embodiments XXXII-XXXIV, wherein the compound is:

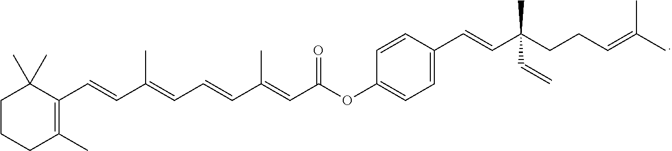

Embodiment XXXVI. A compound having the structure:

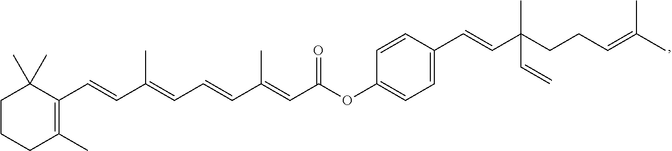 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof, for use in administering an effective amount of bakuchiol and/or retinoic acid to a subject.

Embodiment XXXVII. A compound having the structure:

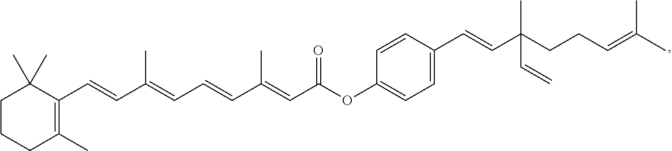 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof, for use in mitigating longitudinal skin sensitivity caused by the continuous administration of retinol or retinoic acid to a subject.

Embodiment XXXVIII. The compound for use of Embodiment XXXVI or XXXVII, wherein the compound is:

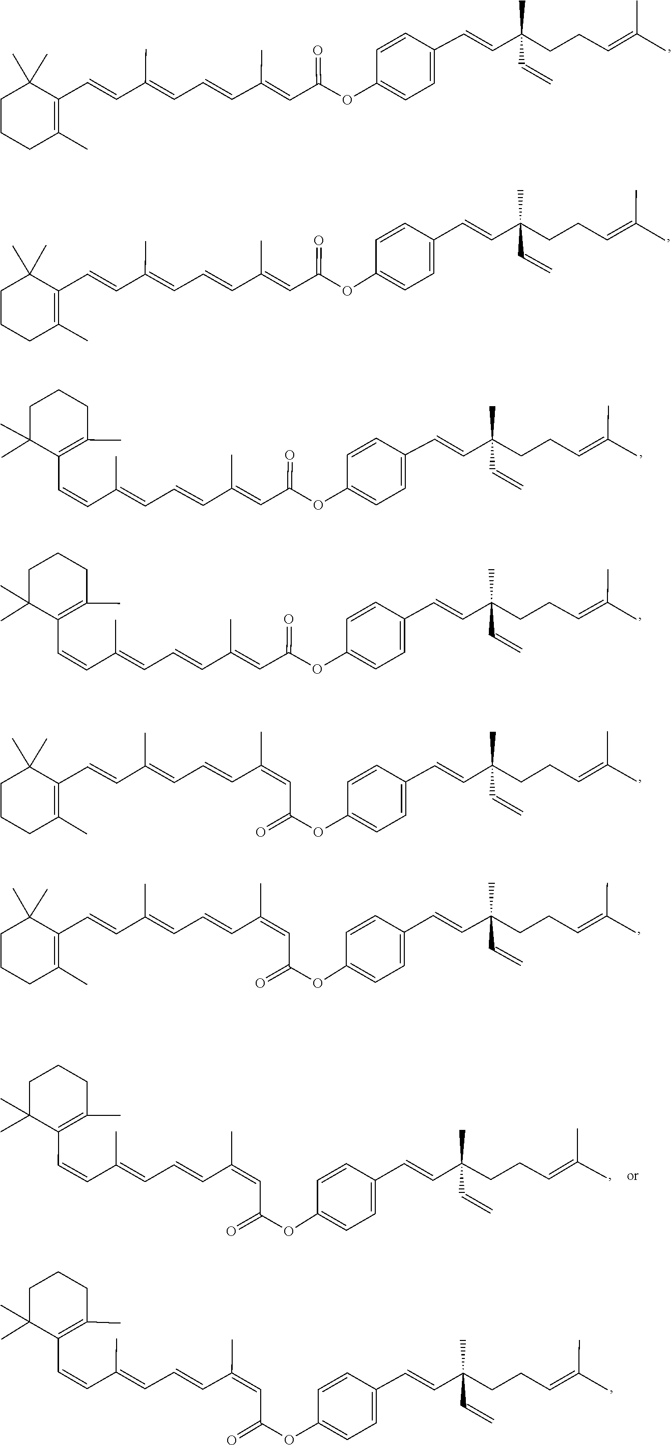

or a mixture of thereof.

Embodiment XXXIX. The compound for use of any one of Embodiments XXXVI-XXXVIII, wherein the compound is:

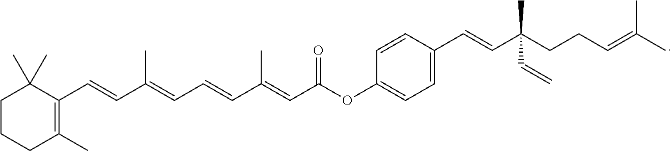

Embodiment XL. A compound having the structure:

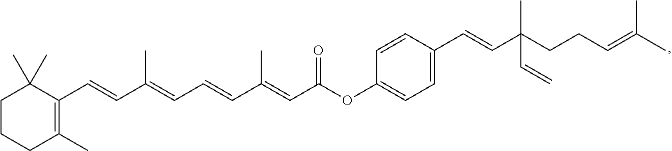 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof, for use in manufacture of a medicament for administering an effective amount of bakuchiol and/or retinoic acid to a subject.

Embodiment XLI. A compound having the structure:

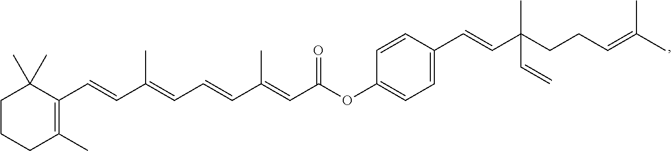 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof, for use in manufacture of a medicament for mitigating longitudinal skin sensitivity caused by the continuous administration of retinol or retinoic acid to a subject.

Embodiment XLII. The compound for use of Embodiment XL or XLI, wherein the compound is:

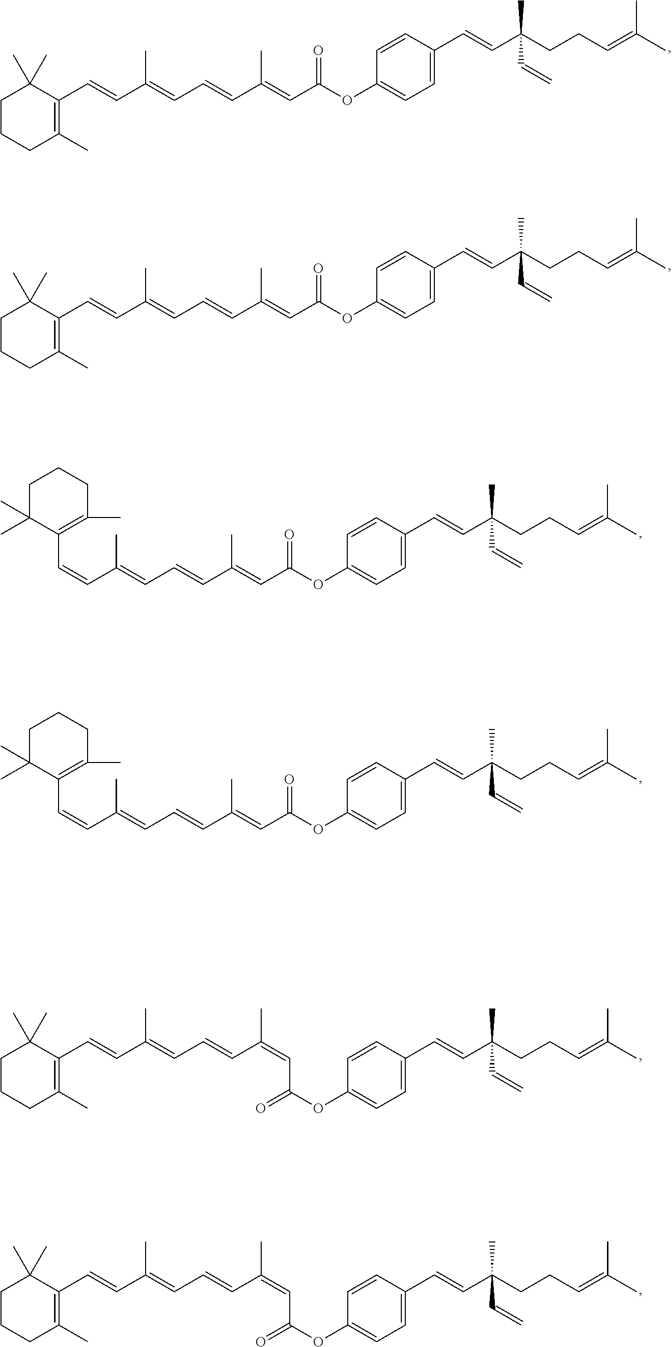

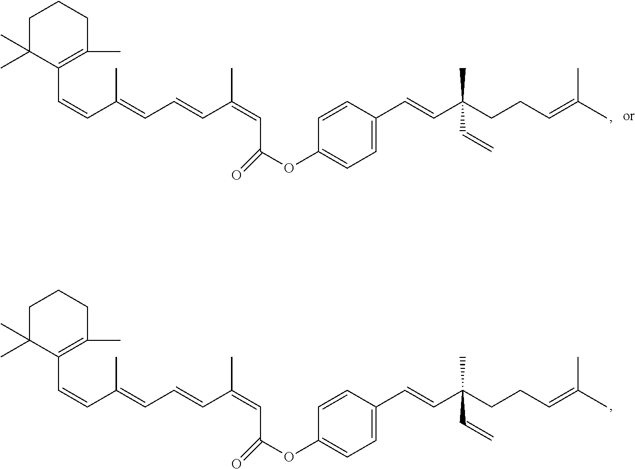

or a mixture thereof.

Embodiment XLIII. The compound for use of any one of Embodiments XL-XLII, wherein the compound is:

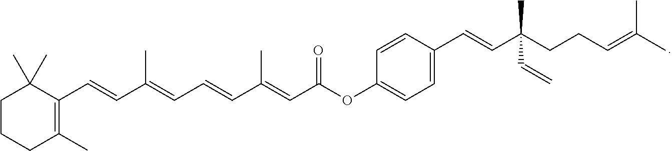

Embodiment XLIV. A composition comprising a compound having the structure:

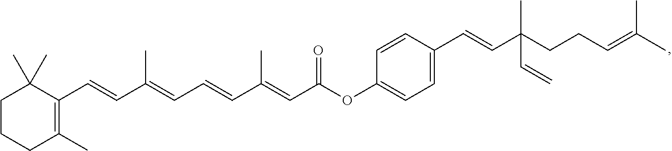 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof, for use in a method of administering an effective amount of bakuchiol and/or retinoic acid to a subject.

Embodiment XLV. A composition comprising a compound having the structure:

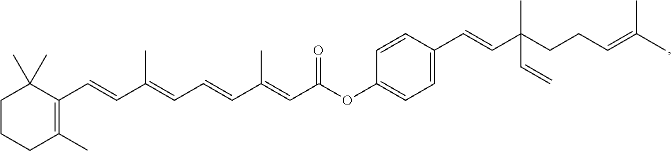 [SMILES: C=CC(C)(/C=C/c1ccc(OC(=O)/C=C(C)/C=C/C=C(C)/C=C/C2=C(C)CCCC2(C)C)cc1)CCC=C(C)C]

or a geometric isomer and/or stereoisomer thereof, or a mixture of isomers thereof, for use in a method of mitigating longitudinal skin sensitivity caused by the continuous administration of retinol or retinoic acid to a subject.

Embodiment XLVI. The composition of Embodiment XLIV or XLV, wherein the compound is:

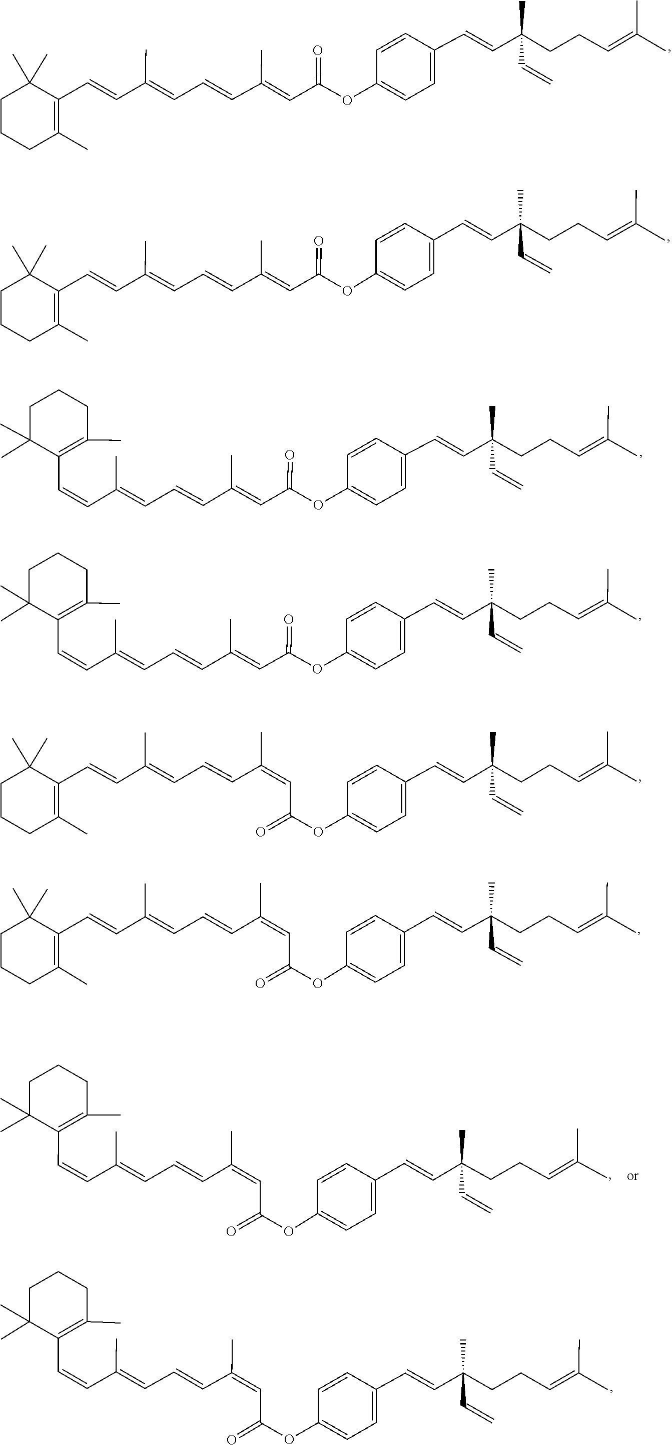

or a mixture thereof.

Embodiment XLVII. The composition of any one of Embodiments XLIV-XLVI, wherein the compound is:

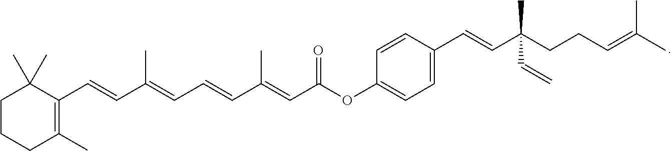

The term “subject” as used herein may be a vertebrate, mammal, or domestic animal. In one embodiment, the subject is a human being.

The terms “treat,” “treating,” “treatment,” and the like as used herein, unless otherwise indicated, refer to eliminating, reducing, or ameliorating a disease or condition, and/or symptoms associated therewith. Although not precluded, treating a disease or condition does not require that the disease, condition, or symptoms associated therewith be completely eliminated. The term “treat” and synonyms contemplate administering a therapeutically effective amount of a Compound of the Disclosure to a subject in need of such treatment. The treatment can be orientated symptomatically, for example, to suppress symptoms. It can be effected over a short period, be oriented over a medium term, or can be a long-term treatment, for example within the context of a maintenance therapy.

The terms “prevent,” “preventing,” and “prevention” as used herein refer to a method of preventing the onset of a disease or condition and/or its attendant symptoms or barring a subject from acquiring a disease. As used herein, “prevent,” “preventing,” and “prevention” also include delaying the onset of a disease and/or its attendant symptoms and reducing a subject's risk of acquiring a disease. The terms “prevent,” “preventing” and “prevention” may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition.

The term “chemical purity” as used herein refers to the extent to which a sample of bakuchinoyl retinoate is free of chemical impurities, e.g. different compounds. For example, if a sample of bakuchinoyl retinoate is said to have a chemical purity of 90%, it may contain up to 10% of different compounds.

The term “isomeric purity” as used herein refers to the extent to which a sample of one isomer of bakuchinoyl retinoate is free of any other isomers, e.g., geometric and/or stereoisomers, of bakuchinoyl retinoate. For example, if a sample of 4-((S,E)-3,7-dimethyl-3-vinylocta-1,6-dien-1-yl)phenyl (2E,4E,6E,8E)-3,7-dimethyl-9-(2,6,6-trimethylcyclohex-1-en-1-yl)nona-2,4,6,8-tetraenoate (Compound 2) is said to have an isomeric purity of 90%, it may contain up to 10% of 4-((R,E)-3,7-dimethyl-3-vinylocta-1,6-dien-1-yl)phenyl (2E,4E,6E,8E)-3,7-dimethyl-9-(2,6,6-trimethylcyclohex-1-en-1-yl)nona-2,4,6,8-tetraenoate (Compound 3).

### EXAMPLES

### Example 1

### Randomized Double-Blind Assessment of Topical Compositions Comprising Compounds of the Disclosure or Retinol for Facial Photoaging

### Study Participants

This study is conducted over a 12-week period as a randomized, double-blinded, rater-blinded study. All participants provide informed written consent prior to participation and receive financial compensation. Healthy participants are recruited and screened for eligibility. Participants are excluded if they are pregnant or breastfeeding, have a known sensitivity to retinol or bakuchiol, or have a cutaneous disease that affects the face. Participants are also excluded if they have used isotretinoin in the previous 6 months, have used a topical antibiotic or topical retinoid in the 30 days prior to enrollment, or have used products containing salicylic acid, β-hydroxy acids or vitamins A, C or E in the last 14 days. Current smokers and those who have smoked within the previous 3 years (as this may serve as a confounder in the assessment of wrinkles) and those who have undergone a facial surgical or cosmetic procedure within 3 months prior to participation are excluded.

### Study Design and Intervention

The study is conducted over 12 weeks and consists of four visits. All treatments are prerandomized using a computer-based randomization generator with blinded allocation via sealed envelopes. Participants are enrolled and assigned interventions by the clinical research coordinator.

The participants are instructed to apply either retinol 0.5% cream to their full face nightly or a 0.5% cream comprising a Compound of the Disclosure, e.g., Compound 2, to their full face twice daily as a thin layer. At each visit the BTBP 3D Clarity Pro® Facial Modeling and Analysis System is utilized to obtain high-resolution facial photographs for all study participants. The photographic instrumentation takes automated photographs in zero ambient lighting with reproducible placement of the face and identical photographic exposures. Participants are also directed to answer a set of subjective tolerability assessment questions of the skin at each follow-up visit. Participants are asked on a scale of 0 (none) to 3 (severe) if they have any itching, burning or stinging. After completion of all study visits, a board-certified dermatologist, blinded to study group assignment, grades scaling, pigmentation and redness.

### Facial Grading and Analysis

Facial photographs are analysed by a computer. In-person grading for pigmentation, erythema and scaling is performed at each visit by a board-certified dermatologist and the same grader is used throughout the study to maintain consistency.

### Tolerability Assessments

Product tolerability is assessed at each follow-up, where participants are asked to grade their experience of itching, burning and stinging along a four-point Likert scale.

### Statistical Analysis

Statistical analyses are performed using paired t-tests (or Wilcoxon signed-rank test for nonparametric measures) with correction for repeated measures with a Bonferroni correction. P-values <0.05 are considered significant, while values between 0.05 and 0.1 are considered a trend.

### Outcomes Measured

The primary outcome measure is image-analysis-based assessment of wrinkle severity and pigmentation at 12 weeks. Secondary outcome measures include image-based analysis of wrinkles and facial pigmentation at earlier time points, and redness, participant-reported tolerability (itching, burning and stinging) and in-person clinical assessments (pigmentation, scaling and erythema) throughout the study.

### Example 2

Formulations Comprising a Compound of the Disclosure Anti-Aging Night Cream with 0.5% Compound of the Disclosure, e.g., Compound 2

| | |
|---|---|
| Component | % w/w |

Phase A1

| | |
|---|---|
| Water | Qs |
| Disodium EDTA | 0.10 |
| Glycerin | 3.00 |
| Butylene Glycol | 2.00 |
| D Panthenol | 0.50 |

Phase A2

| | |
|---|---|
| Xanthan Gum | 0.15 |
| Carbomer | 0.40 |
| Sucrose Stearate | 2.50 |
| Poly C₈-C₂₂ Alkyl Acrylate/Metacrylic | 2.50 |
| Acid Cross Polymer | |

Phase B

| | |
|---|---|
| Simmodsia Chinesis (Jojoba Oil) | 3.00 |
| Shea Butter | 2.50 |
| Behenyl Alcohol | 1.50 |
| Potassium Cetyl Phosphate | 1.00 |
| Stearic Acid | 1.00 |
| Dimethicone | 4.00 |
| Natural Beeswax | 2.00 |
| Isosorbide Dicaprylate | 3.00 |
| Alpha Tocopherol | 0.20 |
| Hydrogenated Polyisobutene | 4.00 |
| Compound of the Disclosure | 0.50 |

Phase C

| | |
|---|---|
| Sodium Hydroxide (50% sol) | 0.40 |

Phase D

| | |
|---|---|
| Fragrance | 0.20 |
| Red 33 (CI 17200) | 0.05 |
| Phenoxyethanol, Ethylhexylglycerine | 1.00 |

The procedure to prepare an anti-aging night cream comprising 0.5% of a Compound of the Disclosure is as follows. The components of Phase A1 are combined. Each component of Phase A2 is individually dispersed in Phase A1 while stirring and heating Phase A1 at 75° C. Phase B is added to Phase A by mixing thoroughly. The mixture is homogenized at moderate speed for 3-5 minutes while adding Phase C, thereby adjusting the pH to 5.5-6.0. The batch is cooled to 40° C. with propeller agitation until the mixture is homogeneous. The components of Phase D are added while continuing to mix the formulation.

Anti-Aging Repair Serum with 0.5% Compound of the Disclosure

| | |
|---|---|
| Component | % w/w |

Phase A1

| | |
|---|---|
| Water | Qs |
| Disodium EDTA | 0.10 |
| Glycerin | 3.00 |
| Butylene Glycol | 2.00 |
| D Panthenol | 0.50 |

Phase A2

| | |
|---|---|
| Acrylates/C₁₀-C₃₀ Alkyl Acrylate | 0.15 |
| Crosspolymer | |

Phase B

| | |
|---|---|
| Triethanolamine | 0.10 |

Phase C

| | |
|---|---|
| Isosorbide Dicaprylate | 2.00 |
| Isostearyl Alcohol, Butylene Glycol | 2.00 |
| Cocoate, Ethylcellulose | |
| Cyclopentasiloxane, Dimethiconol | 2.50 |
| Compound of the Disclosure | 0.50 |

Phase D

| | |
|---|---|
| Hydroxyethylacrylate/sodium | 1.50 |
| Acryloyldimethyltaurate Copolymer, | |
| Squalene, Polysorbate 60 | |

Phase E

| | |
|---|---|
| Poly C₈-C₂₂ Alkyl Acrylate/Metacrylic | 2.00 |
| Acid Cross Polymer | |

Phase F

| | |
|---|---|
| Fragrance | 0.10 |

Phase G

| | |
|---|---|
| Phenoxyethanol, Ethylglycerine | 1.00 |

The procedure to prepare an anti-aging repair serum comprising 0.5% of a Compound of the Disclosure is as follows. The components of Phase A1 are combined. Each component of Phase A2 is individually dispersed in Phase A1 while stirring and heating Phase A1 at 40° C. The components of Phase C are separately combined and heated to 40° C. Phase A is neutralized with Phase B to pH 5.5. Phase C is added to Phase AB by mixing thoroughly. The mixture is homogenized at moderate speed for 3-5 minutes while adding Phase D. The batch is switched to propeller agitation until the mixture is homogeneous. Phases E, F, and G are added while continuing to mix the formulation.

Age-Defying Day Cream with 1% Compound of the Disclosure

| | |
|---|---|
| Component | % w/w |

Phase A1

| Water | Qs |
|---|---|
| Disodium EDTA | 0.10 |
| Glycerin | 2.00 |
| Butylene Glycol | 2.00 |
| D Panthenol | 0.50 |
| Phase A2 | |
| Xanthan Gum | 0.10 |
| Acrylates/C₁₀-C₃₀ Alkyl Acrylate | 0.30 |
| Crosspolymer | |
| Sucrose Stearate | 1.50 |

Phase B

| | |
|---|---|
| Simmodsia Chinensis (Jojoba Oil) | 3.00 |
| Shea Butter | 1.50 |
| Behenyl Alcohol | 1.25 |
| Potassium Cetyl Phosphate | 1.00 |
| Stearic Acid | 1.00 |
| Dimethicone | 5.00 |
| Natural Beeswax | 1.50 |
| Isosorbide Dicaprylate | 2.00 |
| Alpha Tocopherol | 0.20 |
| Ammonium Acryloyldimethyltaurate/VP | 0.30 |
| Copolymer | |

Phase C

| | |
|---|---|
| Sodium Hydroxide (50% sol) | 0.20 |

Phase D

| | |
|---|---|
| Compound of the Disclosure | 1.00 |

Phase E

| | |
|---|---|
| Phenoxyethanol, Ethylhexylglycerine | 1.00 |

Phase F

| | |
|---|---|
| Fragrance | 0.20 |

The procedure to prepare an age-defying day cream comprising 100 of a Compound of the Disclosure is as follows. The components of Phase A1 are combined. Each component of Phase A2 is individually dispersed in Phase A1 while stirring and heating Phase A1 at 75° C. The components of Phase B are separately combined and heated to 75° C. Phase B is added to Phase A by mixing thoroughly. The mixture is homogenized at moderate speed for 3-5 minutes while adding Phase C, thereby adjusting the pH to 5.5-6.0. The batch is cooled while adding Phase D. At 40° C., Phases E and F are added. The batch is mixed with propeller agitation until the mixture is homogeneous. Super Acne-Control Lotion with 1% Compound of the Disclosure and 2% Salicylic Acid

| | |
|---|---|
| Component | % w/w |

Phase A1

| | |
|---|---|
| Water (demineralized) | Qs |
| Disodium EDTA | 0.10 |
| Glycerin | 3.00 |
| Butylene Glycol | 2.00 |
| Salicylic Acid | 2.00 |

Phase A2

| | |
|---|---|
| Xanthan Gum | 0.15 |
| Acrylates/C₁₀-C₃₀ Alkyl Acrylate | 0.15 |
| Copolymer | |

Phase B

| | |
|---|---|
| Sodium Hydroxide (50% sol) | 5.00 |

Phase C

| | |
|---|---|
| Dicaprylyl Ether | 4.00 |
| Cetyl Alcohol | 1.50 |
| Tocopheryl Acetate | 0.20 |
| Glyceryl Stearate, PEG-100 Stearate | 2.00 |
| Cetearyl Alcohol, Ceteareth-20 | 1.00 |
| Dimethicone | 1.00 |
| Cyclomethicone, Polysilicone-11 | 5.00 |
| Beeswax | 1.00 |
| Compound of the Disclosure | 1.00 |

Phase D

| | |
|---|---|
| Polyacrylamide, C₁₃-C₁₄ Isoparaffin, | 1.50 |
| Laureth-4 | |

Phase E

| | |
|---|---|
| Phenoxyethanol, Ethylhexylglycerine | 1.00 |

The procedure to prepare an age-defying day cream comprising 100 of a Compound of the Disclosure and 2% salicylic acid is as follows. The components of Phase A1 are combined. Each component of Phase A2 is individually dispersed in Phase A1 while stirring and heating Phase A1 at 75° C. The components of Phase B are separately combined and heated to 75° C. Phase B is added to Phase A by mixing thoroughly. The mixture is homogenized at high speed while adding Phases C and D. The batch is cooled to 45° C. Phase E is added. The batch stirred gently until the mixture is homogeneous.

| | |
|---|---|
| Component | % w/w |

Phase A

| | |
|---|---|
| Dimethicone, Dimethicol | 30.00 |
| Cyclomethicone | 10.00 |

Phase B

| | |
|---|---|
| Squalane | 3.00 |
| Compound of the Disclosure | 1.00 |

Phase C

| | |
|---|---|
| Cyclomethicone, Polysilicone-11 | 35.00 |
| Cyclomethicone | 20.00 |

The procedure to prepare an anhydrous serum comprising 1% of a Compound of the Disclosure is as follows. All ingredients are mixed in the listed order at room temperature, resulting in a clear translucent serum.

### Example 3

### Synthesis of Bakuchinoyl Retinoate Via Alkyl Chloroformate (100 mg Scale)

Retinoic acid (100 mg, 0.33 mmol) was dissolved in anhydrous tetrahydrofuran (10 ml). Triethylamine (37 mg, 0.37 mmol) was added and the mixture was stirred for five minutes. A solution of isobutyl chloroformate (50 mg, 0.37 mmol) in tetrahydrofuran (1 ml) was added dropwise at 0° C. The mixture was allowed to warm to room temperature and stirred for one hour. Pentane (10 ml) was added and the triethylamine hydrochloride was collected by filtration. The filtrate was evaporated under reduced pressure.

The yellow residue was dissolved in anhydrous acetonitrile (10 ml) and bakuchiol (50 mg, 0.20 mmol) was added. After the mixture was stirred for five minutes, triethylamine (37 mg, 0.37 mmol) and 4-dimethylaminopyridine (10 mg) were added. The mixture was warmed to 50° C. and stirred for one hour. Most of solvent was evaporated under reduced pressure and the residue was mixed with ethyl acetate (20 ml). The ethyl acetate layer was washed with brine (20 ml), dried over magnesium sulfate and evaporated under reduced pressure to give crude product. The crude product was purified by column chromatography on silica gel with hexane/ethyl acetate as the eluent to give 42 mg of pale-yellow oil product. (Molecular Formular: C₃₈H₅₀O₂; Exact Mass:538.38; m/z: 539.39 (M+1)⁺, 561.37 (M+Na)⁺. (H NMR: 400 MHz, DMSO-d₆): 1.00 (s, 6H), 1.17 (s, 3H), 1.42-1.47 (m, 4H), 1.53 (s, 3H), 1.53-1.59 (m, 2H), 1.61 (s, 3H), 1.67 (s, 3H), 1.87-1.93 (m, 2H), 1.99 (t, 2H), 2.00 (s, 3H), 2.34 (s, 3H), 5.00 (d, 1H), 5.04 (d, 1H), 5.10 (t, 1H), 5.90 (dd, 1H), 6.07 (s, 1H), 6.19 (d, 1H), 6.26 (t, 1H), 6.29 (m, 2H), 6.33 (d, 1H), 6.51 (d, 1H), 7.06 (d, 2H), 7.16 (dd, 1H), 7.44 (d, 2H).

### Example 4

### Synthesis of Bakuchinoyl Retinoate via Alkyl Chloroformate (200 mg scale)

Retinoic acid (200 mg, 0.66 mmol) was dissolved in anhydrous tetrahydrofuran (10 ml). Triethylamine (0.2 ml) was added and the mixture was stirred for five minutes. A solution of ethyl chloroformate (72 mg, 0.66 mmol) in tetrahydrofuran (1 ml) was added dropwise at 0° C. The mixture was allowed to warm to room temperature and stirred for one hour. Pentane (10 ml) was added and the triethylamine hydrochloride was collected by filtration. The filtrate was evaporated under reduced pressure.

The yellow residue was dissolved in anhydrous acetonitrile (10 ml) and bakuchiol (50 mg, 0.20 mmol) was added. After the mixture was stirred for five minutes, triethylamine (188 mg, 0.73 mmol) and 4-dimethylaminopyridine (50 mg) were added. The mixture was warmed to 50° C. and stirred for one hour. Ethanolamine (0.2 ml) was added and the mixture was stirred at 50° C. for 30 minutes. Most of solvent was evaporated under reduced pressure and the residue was mixed with ethyl acetate (30 ml). The ethyl acetate layer was washed with 1.0 N hydrochloride solution (30 ml) and brine (30 ml), dried over magnesium sulfate and evaporated under reduced pressure to give crude product, which was purified by column chromatography on silica gel with hexane/ethyl acetate as the eluent to give 258 mg of pale-yellow oil product.

### Example 5

### Synthesis of Bakuchinoyl Retinoate Via Alkyl Chloroformate (3 g Scale)

Retinoic acid (3.00 g, 9.99 mmol) was dissolved in anhydrous tetrahydrofuran (75 ml). Triethylamine (3 ml) was added and the mixture was stirred for five minutes. The solution of ethyl chloroformate (1.08 g, 9.99 mmol) in tetrahydrofuran (10 ml) was added dropwise at 0° C. The mixture was allowed to warm to room temperature and stirred for two hours. Hexane (75 ml) was added and the triethylamine hydrochloride was collected by filtration. The filtrate was evaporated under reduced pressure.

The yellow residue was dissolved in anhydrous acetonitrile (75 ml) and bakuchiol (2.82 g, 10.98 mmol) was added. After the mixture was stirred for five minutes, triethylamine (7.5 ml) and 4-dimethylaminopyridine (1.25 g) were added. The mixture was warmed to 50° C. and stirred for four hours. Ethanolamine (3.0 ml) was added and the mixture was stirred at 50° C. for 30 minutes. Most of solvent was evaporated under reduced pressure and the residue was mixed with ethyl acetate (100 ml). The ethyl acetate layer was washed with 1.0 N hydrochloride solution (100 ml) and brine (100 ml), dried over magnesium sulfate and evaporated under reduced pressure to give crude product, which was purified by column chromatography on silica gel with hexane/ethyl acetate as the eluent to give 3.32 g of pale-yellow oil product.

### Example 6

### Synthesis of Bakuchinoyl Retinoate Via DCC

Retinoic acid (100 mg, 0.33 mmol) and bakuchiol (85 mg, 0.33 mmol) were dissolved in anhydrous dichloromethane (10 ml). Dicyclohexylcarbodiimide (DCC) (103 mg, 0.50 ml) and 4-dimethylaminopyridine (100 mg) were added at 0° C. The mixture was stirred at 0° C. and allowed to warm to room temperature overnight. The reaction mixture was filtered and the filtrate was evaporated under reduced pressure. The residue was purified by column chromatography on silica gel with hexane/ethyl acetate as the eluent to give 108 mg of pale-yellow oil product.

Having now fully described the methods, compounds, and compositions herein, it will be understood by those of skill in the art that the same can be performed within a wide and equivalent range of conditions, formulations, and other parameters without affecting the scope of the methods, compounds, and compositions provided herein or any embodiment thereof. All patents, patent applications, and publications cited herein are fully incorporated by reference herein in their entirety.

1. A compound having the structure: 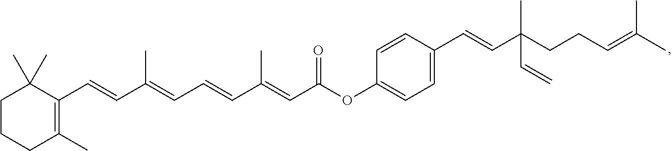 [SMILES: C=CC(C)(/C=C/c1ccc(OC(=O)/C=C(C)/C=C/C=C(C)/C=C/C2=C(C)CCCC2(C)C)cc1)CCC=C(C)C]
or a geometric isomer and/or stereoisomer thereof, or a mixture of isomers thereof.

2. A compound having the structure: 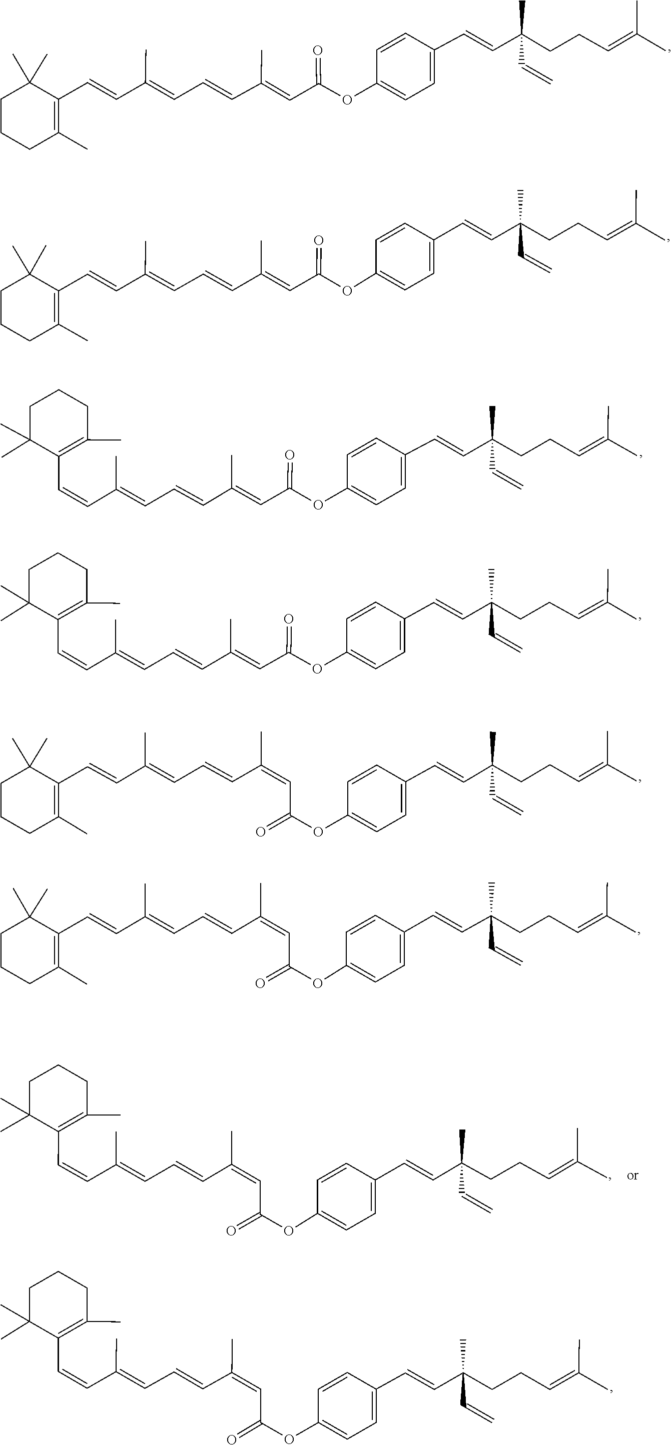
or a mixture thereof.

3. The compound of claim 2, wherein the structure is:

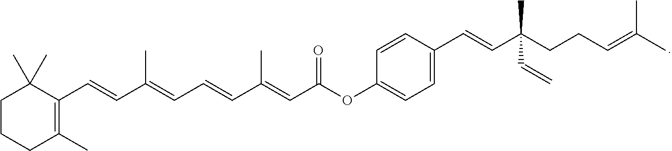

4. A composition comprising the compound of claim 1 and a pharmaceutically, dermatologically, or cosmetically acceptable carrier.

5. The composition of claim 4, wherein the composition further comprises a pH adjuster.

6. The composition of claim 4, wherein the composition further comprises a viscosity modifier.

7. The composition of claim 4, comprising about 0.01% w/w to about 10% w/w of the compound.

8.-10. (canceled)

11. The composition of claim 4, wherein the composition further comprises an effective amount of one or more skin-protective or treatment ingredients.

12. The composition of claim 11, wherein the one or more skin-protective or treatment ingredients are one or more antioxidants, one or more sunscreen actives, one or more skin lightening actives, one or more exfoliants, one or more anti-acne actives, one or more vitamins, one or more anti-inflammatory agents, one or more self-tanning agents, one or more moisturizers, one or more emollients, one or more humectants, or one or more compatible solutes, or a combination thereof.

13. The composition of claim 4, wherein the composition is formulated for topical administration.

14. The composition of claim 4, wherein the composition is formulated as an eye cream or serum, an anti-wrinkle cream or serum, a moisturizing cream, a face wash, a face mask, or a cosmetic.

15.-19. (canceled)

20. A method of improving the common signs of cutaneous facial ageing in a subject, the method comprising administering to the subject an effective amount of a compound of claim 1.

21. (canceled)

22. A method of promoting or maintaining skin health and appearance in a subject, the method comprising administering to the subject an effective amount of a compound of claim 1.

23. A method of preventing, ameliorating, or reducing the loss of softness and elasticity in the skin of a subject, the method comprising administering to the subject an effective amount of a compound of claim 1.

24. A method of preventing, ameliorating, or reducing the presence of fine lines and/or wrinkles in the skin of a subject, the method comprising administering to the subject an effective amount of a compound of claim 1.

25. A method of preventing, ameliorating, or reducing the presence of dark spots in the skin of a subject, the method comprising administering to the subject an effective amount of a compound of claim 1.

26. A method of promoting or maintaining collagen production in the skin of a subject, the method comprising administering to the subject an effective amount of a compound of claim 1.

27. (canceled)

28. A method of administering an effective amount of bakuchiol and/or retinoic acid to a subject, the method comprising administering a compound of claim 1 to the subject.

29. A method of mitigating longitudinal skin sensitivity caused by the continuous administration of retinol or retinoic acid to a subject, the method comprising administering an effective amount of a compound of claim 1 to the subject.

30. (canceled)

31. A method of making the compound of claim 1, or a geometric isomer and/or stereoisomer thereof, or a mixture of isomers thereof, the method comprising:
(a) reacting retinoic acid, or a geometric isomer thereof, with ClC(O)OR, wherein R is a C₁-C₁₀ alkyl group, in solvent to give a carbonic anhydride;
(b) reacting the carbonic anhydride of (a) with bakuchiol, or a geometric isomer and/or stereoisomer thereof, in a solvent to give the compound of claim 1, or a geometric isomer and/or stereoisomer thereof, and, optionally,
(c) isolating the compound of claim 1, or a geometric isomer and/or stereoisomer thereof.
